(19) United States
(12) Patent Application Publication
(10) Pub. No.: US 2024/0112753 A1
(43) Pub. Date: Apr. 4, 2024

(54) TARGET-VARIANT-REFERENCE PANEL FOR IMPUTING TARGET VARIANTS

(71) Applicants: Illumina, Inc., San Diego, CA (US); Illumina Software, Inc., San Diego, CA (US); Illumina Cambridge Limited, Cambridge (GB)

(72) Inventors: Daniel Andrews, Cambridge (GB); Mitchell A. Bekritsky, Decatur, GA (US); Michael A. Eberle, Oceanside, CA (US); Julia Gimbernat Mayol, Cambridge (GB)

(21) Appl. No.: 18/476,206

(22) Filed: Sep. 27, 2023

Related U.S. Application Data
(60) Provisional application No. 63/377,682, filed on Sep. 29, 2022.

(51) Int. Cl. G16B 20/20; G16B 20/10; G16B 20/40; G16B 30/10; G16B 40/20

(52) U.S. Cl. CPC G16B 20/20; G16B 20/10; G16B 20/40; G16B 30/10; G16B 40/20

(57) ABSTRACT

The present disclosure relates to systems, non-transitory computer-readable media, and methods for generating a target-variant-reference panel comprising a target-variant position with target-variant indicators or using the target-variant-reference panel to impute a genotype call for the corresponding target variant. In particular, in one or more embodiments, the disclosed systems generate an initial reference panel including a variety of phased genomic samples of different haplotypes. The disclosed systems further add a target-variant position to the initial reference panel to indicate a presence or absence of a target variant, thereby creating a target-variant-reference panel comprising a target-variant position with target-variant indicators. Additionally or alternatively, the disclosed systems can utilize the target-variant-reference panel to impute genotype calls indicating a presence or absence of a target variant within a target genomic sample based on a comparison of (i) haplotypes represented in the target-variant-reference panel and (ii) nucleotide reads corresponding to the target genomic sample.

### CROSS-REFERENCE TO RELATED APPLICATIONS

The present application claims the benefit of, and priority to, U.S. Provisional Application No. 63/377,682, entitled “A TARGET-VARIANT-REFERENCE PANEL FOR IMPUTING TARGET VARIANTS,” filed on Sep. 29, 2022. The aforementioned application is hereby incorporated by reference in its entirety.

### BACKGROUND

In recent years, biotechnology firms and research institutions have improved hardware and software for sequencing nucleotides and determining nucleobase calls for genomic samples. For instance, some existing sequencing machines and sequencing-data-analysis software (together “existing sequencing systems”) predict individual nucleotides within sequences by using conventional Sanger sequencing or sequencing-by-synthesis (SBS) methods. When using SBS, existing sequencing systems can monitor many thousands of oligonucleotides being synthesized in parallel from templates to predict nucleobase calls for growing nucleotide reads based on images of fluorescently tagged nucleobases incorporated into the oligonucleotides. After capturing such images, some existing sequencing systems determine nucleobase calls for nucleotide reads corresponding to the oligonucleotides and send base-call data to a computing device with sequencing-data-analysis software. By using the sequencing-data-analysis software, existing sequencing systems align nucleotide reads with a reference genome. Based on differences between the aligned nucleotide reads and the reference genome, existing systems can further utilize a variant caller to identify variants of a genomic sample, such as single nucleotide polymorphisms (SNPs), repeat-expansion variants, or insertions or deletions (indels).

Despite these advances, existing sequencing systems frequently determine inaccurate variant calls for difficult-to-call genomic regions, such as regions with variable number tandem repeat (VNTR) expansions, short tandem repeats (STR) expansions, structural variants, or other types of variants. For certain difficult-to-call genomic regions of a genomic sample, existing sequencing systems often use a reference panel and a genotype imputation model to impute nucleobase calls and phase haplotypes based on detected variants in the genomic sample. For instance, existing sequencing systems frequently use various types of hidden Markov models (HMM) customized for imputing genotypes to impute nucleobase calls for certain genomic regions, such as by using Genotype Likelihoods Imputation and PhaSing mEthod (GLIMPSE) or IMPUTE. Based on variants shared among haplotypes of the reference panel and nucleotide reads of a genomic sample, a genotype imputation model can impute variants for difficult-to-call genomic regions of a genomic sample with varying accuracy.

A variant call for a difficult-to-call genomic region can range from inconsequential or critical depending on the gene or other genomic region. Because existing sequencing systems often use reference panels that do not adequately capture or mark variation of repeat-expansion variants (e.g., VNTRs or STRs) or certain pathogenic variants, an incorrect variant call can have significant consequences. For example, a variant call identifying particular repeat-expansion variants in the Replication Factor C Subunit 1 (RFC1) gene can either correctly or incorrectly identify genetic indicators of phenotypes on the Cerebellar Ataxia, Neuropathy, Vestibular Areflexia Syndrome (CANVAS) spectrum. Biallelic intronic AAGGG repeat expansions in the RFC1 gene, for instance, make such variant calls particularly challenging. As a further example, a variant call that correctly or incorrectly identifies a variant for the Cytochrome P450 Family 2 Subfamily D Member 6 (CYP2D6) gene can result in either correctly identifying a genetic indicator of Neuroleptic Malignant Syndrome or miss the genetic indicator entirely. Accordingly, a variant call for such pathogenic variants on a gene may be critical but often lack a suitable reference panel with sufficient variation to support an accurate variant call.

Despite the importance of accurately determining variant calls for repeat expansions and pathogenic variants, existing sequencing systems often cannot generate variant calls or generate inaccurate variant calls because of poor quality nucleotide-read data, poor alignment of nucleotide reads, or inadequate reference panels. Indeed, many existing sequencing systems either do not generate genotype calls or generate inaccurate genotype calls because (i) nucleotide reads corresponding to target genomic regions for target variants provide insufficient coverage, (ii) alignment models cannot accurately map nucleotide reads for such genomic regions on a reference genome, or (iii) existing reference panels include insufficient data to support accurate imputation.

To illustrate the technical problems for (i) and (ii), some existing sequencing systems align nucleotide reads corresponding to a repeat expansion with a target genomic region only to leave read-coverage holes in the middle of the target genomic region. Because target genomic regions for repeat expansions or pathogenic variants can exhibit such read-coverage holes, existing sequencing systems either generate no genotype calls or inaccurate genotype calls. Indeed, without direct evidence from nucleotide reads for a genomic region corresponding to repeat expansions or a reference panel with adequate data for such repeat expansions, existing sequencing systems cannot accurately genotype repeat expansions, such as the repeat expansions in RFC1 and CYP21A2, or other important pathogenic variants.

These along with additional problems and issues exist with regard to existing sequencing systems.

### BRIEF SUMMARY

This disclosure describes one or more embodiments of systems, methods, and non-transitory computer readable storage media that solve one or more of the problems described above or provide other advantages over the art. For example, the disclosed systems can generate a target-variant-reference panel comprising a target-variant position with target-variant indicators or use the target-variant-reference panel to impute a genotype call for the corresponding target variant. More specifically, in one or more embodiments, the disclosed systems generate an initial reference panel including a variety of phased genomic samples of different haplotypes. The disclosed systems further add a target-variant position to the initial reference panel to indicate a presence or absence of a target variant, thereby creating a target-variant-reference panel comprising a target-variant position with target-variant indicators. Additionally or alternatively, the disclosed systems can utilize the target-variant-reference panel to impute genotype calls indicating a presence or absence of a target variant within a target genomic sample based on a comparison of (i) haplotypes represented in the target-variant-reference panel and (ii) nucleotide reads corresponding to the target genomic sample.

Additional features and advantages of one or more embodiments of the present disclosure are outlined in the description which follows, and in part will be obvious from the description, or may be learned by the practice of such example embodiments.

### BRIEF DESCRIPTION OF THE DRAWINGS

The detailed description provides one or more embodiments with additional specificity and detail through the use of the accompanying drawings, as briefly described below.

FIG. 1 illustrates a schematic diagram of a computing system in which a customized genotype-imputation system can operate in accordance with one or more embodiments.

FIG. 2A illustrates the customized genotype-imputation system generating a target-variant-reference panel in accordance with one or more embodiments.

FIG. 2B illustrates the customized genotype-imputation system utilizing a target-variant-reference panel to impute genotype calls in accordance with one or more embodiments.

FIG. 3 illustrates nucleotide reads of a genomic sample misaligned with a genomic region comprising a repeat expansion in accordance with one or more embodiments.

FIG. 4 illustrates a clustering pattern of genomic samples including a target variant in accordance with one or more embodiments.

FIG. 5 illustrates the customized genotype-imputation system generating a target-variant-reference panel including a target-variant position in accordance with one or more embodiments.

FIG. 6 illustrates an example output file comprising a target-variant-reference panel in accordance with one or more embodiments.

FIG. 7 illustrates a graph depicting non-reference-genotype-concordance rates for the customized genotype-imputation system using a target-variant-specific target-variant-reference panel relative to allele frequency in accordance with one or more embodiments.

FIG. 8 illustrates the customized genotype-imputation system imputing genotype calls for a target variant within a genomic sample utilizing a target-variant-reference panel in accordance with one or more embodiments.

FIG. 9 illustrates a graphical user interface for providing information about imputed genotype calls for target variants in accordance with one or more embodiments.

FIG. 10 illustrates a flowchart of a series of acts for generating a target-variant-reference panel in accordance with one or more embodiments.

FIG. 11 illustrates a flowchart of a series of acts for utilizing a target-variant-reference panel to impute genotype calls in accordance with one or more embodiments.

FIG. 12 illustrates a block diagram of an example computing device for implementing one or more embodiments of the present disclosure.

### DETAILED DESCRIPTION

This disclosure describes one or more embodiments of a customized genotype-imputation system that generates a target-variant-reference panel including a target-variant position for target-variant indicators or utilizes the target-variant-reference panel to impute genotype calls for the corresponding target variant. To illustrate, in one or more embodiments, the customized genotype-imputation system creates an initial reference panel including genomic samples of genetically diverse haplotypes. The customized genotype-imputation system further adds a target-variant position to the initial reference panel and phases alleles of the genomic samples to determine a presence or absence of the target variant in corresponding alleles present on maternal haplotypes and paternal haplotypes. By adding such a target-variant position, the customized genotype-imputation system generates a target-variant-reference panel comprising target-variant indicators within the target-variant position for the phased alleles of the genomic samples. Having generated or accessed such a target-variant-reference panel, in one or more embodiments, the customized genotype-imputation system utilizes the target-variant-reference panel to determine genotype calls indicating a presence or absence of a target variant within a target genomic sample.

As mentioned, in one or more embodiments, the customized genotype-imputation system generates a target-variant-reference panel. To generate the target-variant-reference panel, in one or more embodiments, the customized genotype-imputation system generates an initial reference panel including genomic samples with genetically diverse haplotypes. To illustrate, in one or more embodiments, the customized genotype-imputation system generates the initial reference panel including genomic samples from various populations, ancestries, continents, and/or countries. In some embodiments, the haplotypes in the initial reference panel include one or more marker variants, such as single nucleotide polymers (SNPs) or small insertions and/or deletions.

Based on the initial reference panel, in some implementations, the customized genotype-imputation system generates the target-variant-reference panel by adding a target-variant position to the initial reference panel. For example, in some embodiments, the customized genotype-imputation system adds a data field as a placeholder for an indicator of a target variant present in alleles of the various haplotypes represented in the initial reference panel. In one or more embodiments, the customized genotype-imputation system inserts a target-variant indicator into such a data field (or another target-variant position) to indicate whether a given genomic sample includes the target variant. In contrast to conventional reference panels that do not include such a target-variant position, the customized genotype-imputation system can utilize the target-variant position of a target-variant-reference panel to identify target variants more accurately.

In addition to adding a target-variant position, in some cases, the customized genotype-imputation system phases the alleles of the genomic samples represented by the target-variant-reference panel based on SNPs or other marker variants exhibited by various haplotypes' alleles. To illustrate, in some embodiments, the customized genotype-imputation system utilizes a haplotype phasing model to phase the alleles of the genomic samples based on known haplotypes and other inheritance patterns. More specifically, in one or more embodiments, the customized genotype-imputation system (i) identifies one or more genomic coordinates corresponding to a target variant and (ii) phases alleles from the haplotypes corresponding to those genomic coordinates based on marker variants exhibited by the alleles. By phasing the alleles of the genomic samples with indicators in a target-variant position, the customized genotype-imputation system can include a target-variant indicator for a target variant specific to phased alleles of various haplotypes in the target-variant-reference panel. As explained below, the customized genotype-imputation system can utilize a variety of other phasing models to phase the alleles of genomic samples represented by the target-variant-reference panel.

In addition or in the alternative to generating a target-variant-reference panel, in one or more embodiments, the customized genotype-imputation system utilizes the target-variant-reference panel to impute one or more genotype calls for a target variant of a target genomic sample. To illustrate, in one or more embodiments, the customized genotype-imputation system receives and/or identifies nucleotide reads corresponding to a target genomic sample. The customized genotype-imputation system further accesses a target-variant-reference panel comprising target-variant indicators within a target-variant position for phased alleles of genomic samples of different haplotypes. Based on comparing alleles of the haplotypes represented by the target-variant-reference panel to the nucleotide reads corresponding to the target genomic sample, in some embodiments, the customized genotype-imputation system imputes a genotype call for the target variant within the target genomic sample.

For example, in one or more embodiments, a sequencing device receives nucleotide-sample slide (e.g., flow cell) comprising oligonucleotides extracted from a target genomic sample and determines nucleotide reads corresponding to the oligonucleotides for the target genomic sample. In addition, or in the alternative, the customized genotype-imputation system can receive data representing nucleotide reads for a target genomic sample. In some cases, the customized genotype-imputation system receives nucleotide reads for the target genomic sample from a third-party sequencing system.

As mentioned, in one or more embodiments, the customized genotype-imputation system compares reads of the target genomic sample to alleles of genomic samples included in the target-variant-reference panel. To illustrate, the customized genotype-imputation system can identify marker variants in the target sample surrounding one or more genomic coordinates corresponding to the target variant. The customized genotype-imputation system further compares marker variants indicated by nucleotide reads of the target genomic sample to corresponding marker variants within the haplotypes' alleles in the target-variant-reference panel. In some cases, the customized genotype-imputation system phases the nucleotide reads of the target genomic sample to identify corresponding alleles in maternal and paternal haplotypes in the target-variant-reference panel.

Based on comparing alleles of the haplotypes represented by the target-variant-reference panel to the nucleotide reads corresponding to the target genomic sample, the customized genotype-imputation system generates a prediction of whether the target genomic sample carries the target variant. To illustrate, in some cases, the customized genotype-imputation system determines a phased genotype call indicating the presence or absence of the target variant at an allele corresponding to a maternal or paternal haplotype. Accordingly, the customized genotype-imputation system can determine whether the target genomic sample is a carrier of a target variant at a particular allele, a case of the target variant at both alleles, or unaffected by the target variant at either allele. Thus, in one or more embodiments, the customized genotype-imputation system can generate and provide a notification or graphics indicating a phased genotype call within a graphical user interface via a computing device.

As suggested above, the customized genotype-imputation system provides several technical advantages and benefits over existing sequencing systems and methods. For example, the customized genotype-imputation system improves accuracy of genotype calling for target variants. By generating or utilizing a target-variant-reference panel to impute genotype calls for a target variant corresponding to haplotypes for a genomic sample, the customized genotype-imputation system improves the accuracy of imputation for target variants, especially for difficult-to-call genomic regions exhibiting repeat expansions or other variant types. To illustrate, by utilizing the target-variant-reference panel comprising a target-variant position, the customized genotype-imputation system can generate accurate and phased genotype calls for target variants in genomic regions of a reference genome with which nucleotide reads are difficult to align, including genomic regions where many existing sequencing systems cannot generate any genotype call or cannot generate accurate genotype calls. For example, the customized genotype-imputation system can generate accurate genotype calls for repeat expansions in the RFCJ gene, CYP2D6 gene, or various other genes referenced below, in part by generating or using a target-variant-reference panel that includes both marker variants and a target-variant position with target-variant indicators for particular genomic samples.

The customized genotype-imputation system improves genotype calling by utilizing a first-of-its-kind reference panel. More specifically, the customized genotype-imputation system generates or utilizes a target-variant-reference panel that is customized with target-variant positions specific to one or more target variants. No existing reference panels include target-variant positions with target-variant indicators of a presence or absence of target variants on maternal and paternal haplotypes. The disclosed target-variant-reference panel facilitates more accurate genotype calls—including more accurate phased genotype calls—for repeat expansions and other pathogenic variants by enabling the customized genotype-imputation system to compare nearby marker variants within nucleotide reads of a target genomic sample and alleles of haplotypes represented by the target-variant-reference panel with corresponding target-variant indicators.

In addition to improved genotype calling for target variants, in one or more embodiments, the customized genotype-imputation system improves computer-processing efficiency and uses less memory relative to existing reference panels by generating a target-variant-reference panel that includes data for one or more target genomic regions (or genomic regions of interest) corresponding to a target variant. To illustrate, in some embodiments, the customized genotype-imputation system limits a target-variant-reference panel to include data representing haplotypes of genomic samples corresponding to one or more target genomic regions corresponding to a target variant, but not data representing haplotypes outside the one or more target genomic regions. This improves efficiency and conserves computing resources by reducing or eliminating excess analysis of other genomic coordinates performed by conventional systems. Because some existing reference panels can include a haplotype matrix with 50 million cells representing different marker variants and haplotypes—and existing sequencing systems can determine 40,000 genotype calls based on 40,000 haplotype matrices within reference panels—a relatively small reduction in the size of a target-variant-reference panel can result in considerable memory and computer-processing savings. By reducing or eliminating unnecessary genomic regions and using a target-variant-reference panel comprising data limited to one or more target genomic regions, the customized genotype-imputation system uses less memory and expedites the computer-processing time for imputing genotype calls for target variants.

As illustrated by the foregoing discussion, the present disclosure utilizes a variety of terms to describe features and advantages of the customized genotype-imputation system. Additional detail is now provided regarding the meaning of such terms. For example, as used herein, the term “nucleotide read” (or simply “read”) refers to an inferred sequence of one or more nucleotide bases (or nucleobase pairs) from all or part of a sample nucleotide sequence. In particular, a nucleotide read includes a determined or predicted sequence of nucleobase calls for a nucleotide fragment (or group of monoclonal nucleotide fragments) from a sequencing library corresponding to a genomic sample. For example, in some cases, a sequencing device determines a nucleotide read by generating nucleobase calls for nucleobases passed through a nanopore of a nucleotide-sample slide, determined via fluorescent tagging, or determined from a well in a flow cell.

Additionally, as used herein, the term “nucleobase call” (or sometimes simply “base call”) refers to a determination or prediction of a particular nucleotide base (or nucleotide pair) for a genomic coordinate of a sample genome or for an oligonucleotide during a sequencing cycle or for a genomic coordinate of a sample genome. In particular, a nucleobase call can indicate (i) a determination or prediction of the type of nucleobase that has been incorporated within an oligonucleotide on a nucleotide-sample slide (e.g., read-based nucleobase calls) or (ii) a determination or prediction of the type of nucleobase that is present at a genomic coordinate or region within a genome, including a variant call or a non-variant call in a digital output file. In some cases, for a nucleotide read, a nucleobase call includes a determination or a prediction of a nucleobase based on intensity values resulting from fluorescent-tagged nucleotides added to an oligonucleotide of a nucleotide-sample slide (e.g., in a cluster of a flow cell). Alternatively, a nucleobase call includes a determination or a prediction of a nucleobase from chromatogram peaks or electrical current changes resulting from nucleotides passing through a nanopore of a nucleotide-sample slide. By contrast, a nucleobase call can also include a final prediction of a nucleobase at a genomic coordinate of a sample genome for a variant call file (VCF) or other base-call-output file-based on nucleotide reads corresponding to the genomic coordinate. Accordingly, a nucleobase call can include a base call corresponding to a genomic coordinate and a reference genome, such as an indication of a variant or a non-variant at a particular location corresponding to the reference genome. Indeed, a nucleobase call can refer to a variant call, including but not limited to, a single nucleotide variant (SNV), an insertion or a deletion (indel), or base call that is part of a structural variant. As suggested above, a single nucleobase call can be an adenine (A) call, a cytosine (C) call, a guanine (G) call, or a thymine (T) call.

Further, as used herein, the term “variant” refers to one or more nucleobase calls that differs or varies from a reference base (or reference bases) of a reference genome. To illustrate, a variant nucleobase call can include (or be part of) various structural variant that differ from one or more reference bases of a reference genome. To illustrate, a variant can include an SNP, a deletion, an insertion, a duplication, an inversion, a translocation, or a copy number variation (CNV). In one or more embodiments, a variant comprises a mutation, including a naturally or synthetically introduced mutation, such as a CRISPR-induced mutation.

Relatedly, as used herein, the term “target variant” refers to a variant that is selected or identified for detection or imputation. In some cases, a target variant includes a variant that a variant caller, variant calling model, or other caller has identified for detection. For instance, a target variant may be identified by a repeat expansion detection model, a structural variant caller, a CYP2D6 caller, a CNV caller, a small variant caller, or other caller for detection. As noted below, a target variant may be a variant for a particular gene, including, but not limited to, a Replication Factor C Subunit 1 (RFC1) gene, a Cytochrome P450 Family 2 Subfamily D Member 6 (CYP2D6) gene, Cytochrome P450 Family 2 Subfamily B Member 6 (CYP2B6) gene, Cytochrome P450 Family 21 Subfamily A Member 2 (CYP21A2) gene, Survival Motor Neuron 1 (SMN1) gene, Survival Motor Neuron 2 (SMN2) gene, Glucosylceramides Beta (GBA) gene, Blood Group Rh(CE) (RHCE) gene, Lipoprotein(A) (LPA) gene, a Fragile X Mental Retardation 1 (FMR1) gene, a Hexosaminidase Subunit Alpha (HEXA) gene, Hemoglobin Subunit Alpha 1 (HBA1) gene, Hemoglobin Subunit Alpha 2 (HBA2) gene, or a Hemoglobin Subunit Beta (HBB) gene.

Further, as used herein, the term “impute” refers to statistically inferring or estimating a genotype for a genomic coordinate or a genomic region. More specifically, imputing can include statistically inferring a genotype for one or more alleles corresponding to haplotypes for a genomic region of a sample genome. For example, imputing can refer to utilizing marker variants surrounding a genomic region to determine genotypes for alleles corresponding to haplotypes for the genomic region. In one or more embodiments, the customized genotype-imputation system utilizes reference panels from a haplotype database and a genotype imputation model (e.g., Hidden Markov-based model) to impute genotype calls. As described further herein, the customized genotype-imputation system can impute genotype calls for a target variant within a target genomic region based on SNPs (or other marker variants) that surround or flank the target genomic region but are also part of one or more haplotypes corresponding to the target genomic region. For instance, if haplotypes exhibit different sets of SNPs in a target genomic region and some genomic samples in a target-variant-reference panel also exhibit a target variant, the customized genotype-imputation system can use such different sets of SNPs and target-variant indicators corresponding to certain haplotypes of the genomic sample to infer a target genomic sample comprises the target variant.

As used herein, the term “reference genome” refers to a digital nucleic acid sequence assembled as a representative example (or representative examples) of genes and other genetic sequences of an organism. Regardless of the sequence length, in some cases, a reference genome represents an example set of genes or a set of nucleic acid sequences in a digital nucleic acid sequence determined as representative of an organism. For example, a linear human reference genome may be GRCh38 (or other versions of reference genomes) from the Genome Reference Consortium. GRCh38 may include alternate contiguous sequences representing alternate haplotypes, such as SNPs and small indels (e.g., 10 or fewer base pairs, 50 or fewer base pairs).

Also, as used herein, the term “reference panel” refers to a digital collection or database of haplotypes from genomic samples for which one or more ancestral or progenitorial haplotypes have been determined. In some cases, a reference panel includes a digital database of haplotypes from genomic samples representative of (or common among) an organism's population and for which multiple ancestral or progenitorial haplotypes have been determined. A reference panel can likewise include a data file or other organization of data reflecting genomic sequences and various variant markers (e.g., SNPs) in those genomic sequences. To illustrate, a reference panel can include data corresponding to genomic sequences and various tags or other metadata characterizing or categorizing the genomic sequences. In some cases, the customized genotype-imputation system accesses an initial reference panel developed by the Haplotype Reference Consortium (HRM), 1000 Genomes Project, or Illumina, Inc. when generating a reference panel comprising marker-variant indicators for marker variants at genomic coordinates corresponding to genomic samples of different haplotypes.

Further, used herein, the term “target-variant-reference panel” refers to a reference panel comprising data for genomic sequences from genomic samples of different haplotypes and one or more target-variant positions comprising target-variant indicators for one or more target variants. In particular, a target-variant-reference panel can include genomic sequences including data indications for various marker variants (e.g., SNPs) and data fields for indicating the presence or absence of one or more target variants. To illustrate, a target-variant-reference panel can include diverse genomic samples phased into maternal and paternal sequences and data fields representing target-variant positions that indicate the presence or absence of target variants for both paternal and maternal genomic sequences.

Relatedly, as used herein, the term “target-variant position” refers to a data attribute, characteristic, cell, or field for indicators of a target variant. In particular, the term target-variant position can include a data cell or a data field in which a target-variant indicator can be added or inserted to identify a presence or absence of a target variant in an allele, a haplotype, or a genomic sample. To illustrate, a target-variant position can include a data field in a target-variant-reference panel in which a “0” indicates the absence of a target variant and/or where a “1” indicates the presence of a target variant. In some cases, a target-variant-reference panel includes a target-variant position for target-variant indicators of a biallelic target variant. In addition or in the alternative, in some embodiments, a target-variant-reference panel may include multiple target-variant positions that include multiple data entries or other target-variant indicators for a multi-allelic target variant.

Additionally, as used herein, the term “marker variant” refers to a variant at a polymorphic site in a population. In particular, a marker variant includes one of two or more alleles present among a population at a polymorphic genomic coordinate or genomic region at a frequency greater than a threshold frequency, such as greater than 1% of a population. In some cases, a marker variant includes SNPs present at a polymorphic genomic coordinate among a human population that is represented in a reference panel. Additionally, or alternatively, a marker variant can include insertions or deletions (indels), structural variants, or other variants at polymorphic sites among a population. As suggested above, alleles for particular haplotypes represented by a reference panel may include SNPs or other variant markers used for imputation.

Relatedly, as used herein, the term “marker-variant indicator” refers to a data indication of a marker variant. Similarly, as also used herein, the term “target-variant indicator” refers to a data indication of a target variant. In particular, the term marker-variant indicator or marker-variant indicator can include a “1” in a file (e.g., VCF) indicating the presence of a variant at a particular genomic coordinate or a “0” in the file reflecting the absence of a variant at a particular genomic coordinate. However, it will be understood that a marker-variant indicator and/or a target-variant indicator can include another data indications reflecting the presence or absence of a variant, such as single-letter codes, alphanumeric codes, or other symbols.

Additionally, as used herein, the term “genomic coordinate” refers to a particular location or position of a nucleotide base within a genome (e.g., an organism's genome or a reference genome). In some cases, a genomic coordinate includes an identifier for a particular chromosome of a genome and an identifier for a position of a nucleotide base within the particular chromosome. For instance, a genomic coordinate or coordinates may include a number, name, or other identifier for a chromosome (e.g., chr1 or chrX) and a particular position or positions, such as numbered positions following the identifier for a chromosome (e.g., chr1:1234570 or chr1:1234570-1234870). Further, in certain implementations, a genomic coordinate refers to a source of a reference genome (e.g., mt for a mitochondrial DNA reference genome or SARS-CoV-2 for a reference genome for the SARS-CoV-2 virus) and a position of a nucleotide-base within the source for the reference genome (e.g., mt:16568 or SARS-CoV-2:29001). By contrast, in certain cases, a genomic coordinate refers to a position of a nucleotide-base within a reference genome without reference to a chromosome or source (e.g., 29727).

Also, as used herein, the term “genomic region” refers to a range of genomic coordinates. Like genomic coordinates, in certain embodiments, a genomic region may be identified by an identifier for a chromosome and a particular position or positions, such as numbered positions following the identifier for a chromosome (e.g., chr1:1234570-1234870).

Relatedly, the term “target genomic region” refers to a genomic region that includes a target variant and nucleobases that surround or flank the target variant. In particular, a target genomic region can include the genomic coordinate(s) for a target variant and at least genomic coordinates for marker variants within a threshold number of nucleobases (e.g., 50 base pairs, 200 base pairs, 500 base pairs, 1,000 base pairs) upstream of the target genomic region and/or within a threshold number of nucleobases (e.g., 50 base pairs, 200 base pairs, 500 base pairs, 1,000 base pairs) downstream from the target genomic region.

As also used herein, the term “haplotype” refers to nucleotide sequences that are present in an organism (or present in organisms from a population) and inherited from one or more ancestors. In particular, a haplotype can include alleles or other nucleotide sequences present in organisms of a population and inherited together by such organisms respectively from a single parent. In one or more embodiments, haplotypes include a set of SNPs on the same chromosome that tend to be inherited together. In some cases, data representing a haplotype or a set of different haplotypes are stored or otherwise accessible on a haplotype database.

Further, as used herein, the term “genomic sample” refers to a target genome or portion of a genome undergoing sequencing. For example, a sample genome includes a sequence of nucleotides isolated or extracted from a sample organism (or a copy of such an isolated or extracted sequence). In particular, a sample genome includes a full genome that is isolated or extracted (in whole or in part) from a sample organism and composed of nitrogenous heterocyclic bases. A sample genome can include a segment of deoxyribonucleic acid (DNA), ribonucleic acid (RNA), or other polymeric forms of nucleic acids or chimeric or hybrid forms of nucleic acids noted below. In some cases, the sample genome is found in a sample prepared or isolated by a kit and received by a sequencing device.

Relatedly, the term “allele” refers to a version of a nucleobase or nucleotide sequence at a genomic coordinate or genomic region corresponding to a haplotype, such as a haplotype for a genomic region encoding for a gene or a non-coding region. In particular, an allele includes one of two or more versions of a nucleobase or a nucleotide sequence at a genomic coordinate or region that tend to be inherited together in combination as part of a haplotype. As part of a haplotype, in some cases, a combination of alleles may be inherited by an organism as part of a single gene or across multiple genes.

Additionally, as used herein, the term “genetic diversity” refers to a range of different inherited variants within a population. In particular, genetic diversity includes a range of inherited variants exhibited by different haplotypes representing different ancestries, continents, countries, and/or populations. More specifically, a reference panel can include data representing haplotypes exhibiting genetic diversity among the variants within the haplotypes' alleles.

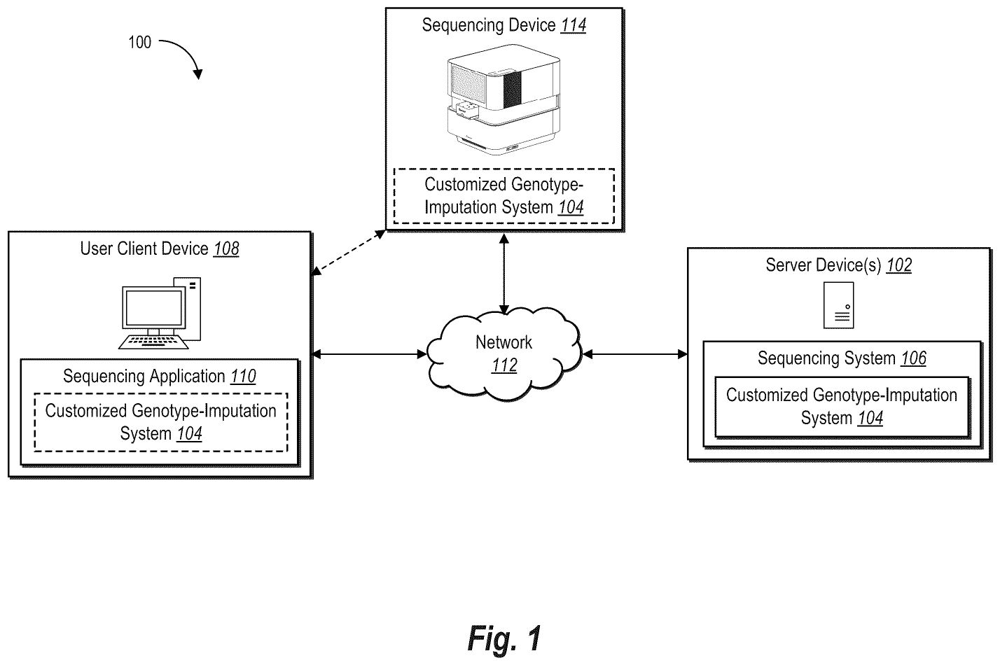

Additional detail will now be provided in relation to illustrative figures portraying example embodiments and implementations of the persona group system. For example, FIG. 1 illustrates a schematic diagram of a computing system 100 in which a customized genotype-imputation system 104 and a sequencing system 106 operate in accordance with one or more embodiments. As illustrated, the computing system 100 includes one or more server device(s) 102 connected to a user client device 108 and a sequencing device 114 via a network 112. While FIG. 1 shows an embodiment of the customized genotype-imputation system 104, this disclosure describes alternative embodiments and configurations below.

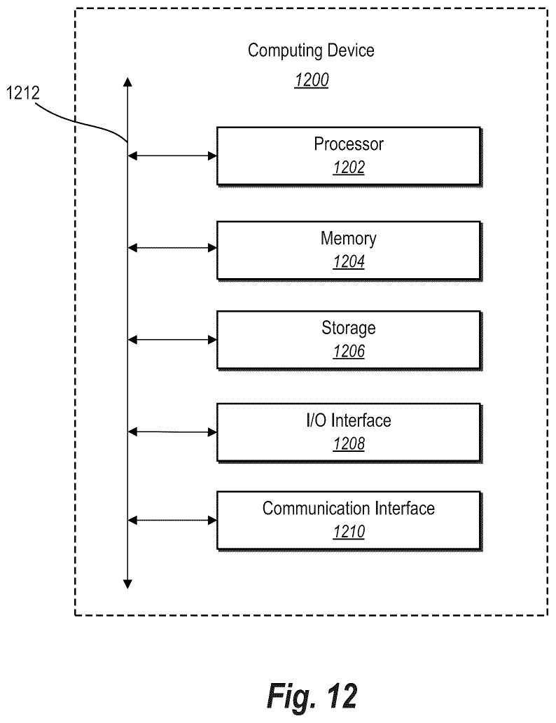

As shown in FIG. 1, the server device(s) 102, the user client device 108, and the sequencing device 114 are connected via the network 112. Accordingly, each of the components of the computing system 100 can communicate via the network 112. The network 112 comprises any suitable network over which computing devices can communicate. Example networks are discussed in additional detail below with respect to FIG. 12.

As indicated by FIG. 1, the sequencing device 114 comprises a device for sequencing a genomic sample or other nucleic-acid polymer. In some embodiments, the sequencing device 114 analyzes oligonucleotides extracted from genomic samples to generate data utilizing computer implemented methods and systems (described herein) either directly or indirectly on the sequencing device 114. More particularly, the sequencing device 114 receives and analyzes, within nucleotide-sample slides (e.g., flow cells), nucleic-acid sequences extracted from genomic samples. In one or more embodiments, the sequencing device 114 utilizes SBS to sequence a genomic sample or other nucleic-acid polymers. In addition, or in the alternative to communicating across the network 112, in some embodiments, the sequencing device 114 bypasses the network 112 and communicates directly with the user client device 108. Additionally, as shown in FIG. 1, in one or more embodiments, the sequencing device 114 includes the customized genotype-imputation system 104.

As further indicated by FIG. 1, the server device(s) 102 may generate, receive, analyze, store, and transmit digital data, such as data for nucleobase calls or nucleotide reads. As shown in FIG. 1, the sequencing device 114 may send (and the server device(s) 102 may receive) various data from the sequencing device 114, including data representing nucleotide reads. The server device(s) 102 may also communicate with the user client device 108. In particular, the server device(s) 102 can send data for nucleotide reads, nucleobase calls, genomic samples, and/or reference panels to the user client device 108. Additionally, as shown in FIG. 1, the server device(s) 102 can include the customized genotype-imputation system 104. In one or more embodiments, as explained further below, the customized genotype-imputation system 104 generates a target-variant-reference panel including one or more target-variant positions. Accordingly, the server device(s) 102 can also send data representing a target-variant-reference panel to the user client device 108.

In some embodiments, the server device(s) 102 comprise a distributed collection of servers where the server device(s) 102 include a number of server devices distributed across the network 112 and located in the same or different physical locations. Further, the server device(s) 102 can comprise a content server, an application server, a communication server, a web-hosting server, or another type of server.

In some cases, the server device(s) 102 is located at or near a same physical location of the sequencing device 114 or remotely from the sequencing device 114. Indeed, in some embodiments, the server device(s) 102 and the sequencing device 114 are integrated into a same computing device. The server device(s) 102 may run the sequencing system 106 or the customized genotype-imputation system 104 to generate, receive, analyze, store, and transmit digital data, such as by receiving base-call data or determining variant calls based on analyzing such base-call data.

As further illustrated and indicated in FIG. 1, the user client device 108 can generate, store, receive, and send digital data. In particular, the user client device 108 can receive data for the nucleotide reads, nucleobase calls, genotype calls, sequencing metrics, and/or target-variant-reference panels from the server device(s) 102 and/or the sequencing device 114. The user client device 108 can accordingly present data concerning genotype calls within a graphical user interface to a user associated with the user client device 108.

The user client device 108 illustrated in FIG. 1 may comprise various types of client devices. For example, in some embodiments, the user client device 108 includes non-mobile devices, such as desktop computers or servers, or other types of client devices. In yet other embodiments, the user client device 108 includes mobile devices, such as laptops, tablets, mobile telephones, or smartphones. Additional details with regard to the user client device 108 are discussed below with respect to FIG. 12.

As further illustrated in FIG. 1, the user client device 108 includes a sequencing application 110. The sequencing application 110 may be a web application or a native application stored and executed on the user client device 108 (e.g., a mobile application, desktop application). The sequencing application 110 can include instructions that (when executed) cause the user client device 108 to receive data from the customized genotype-imputation system 104 and present data from the sequencing device 114 and/or the server device(s) 102. Furthermore, the sequencing application 110 can instruct the user client device 108 to display data for genotype calls, such as genotype calls for a target variant from a variant call file (VCF).

As further illustrated in FIG. 1, the customized genotype-imputation system 104 may be located on the user client device 108 as part of the sequencing application 110 or on the sequencing device 114. Accordingly, in some embodiments, the customized genotype-imputation system 104 is implemented by (e.g., located entirely or in part) on the user client device 108. As mentioned, in yet other embodiments, the customized genotype-imputation system 104 is implemented by one or more other components of the computing system 100, such as the sequencing device 114. In particular, the customized genotype-imputation system 104 can be implemented in a variety of different ways across the server device(s) 102, the network 112, the user client device 108, and the sequencing device 114.

Though FIG. 1 illustrates the components of the computing system 100 communicating via the network 112, in certain implementations, the components of the computing system 100 can also communicate directly with each other, bypassing the network. For instance, and as previously mentioned, in some implementations, the user client device 108 communicates directly with the sequencing device 114. Additionally, in some embodiments, the user client device 108 communicates directly with the customized genotype-imputation system 104. Moreover, the customized genotype-imputation system 104 can access one or more databases housed on or accessed by the server device(s) 102 or elsewhere in the computing system 100.

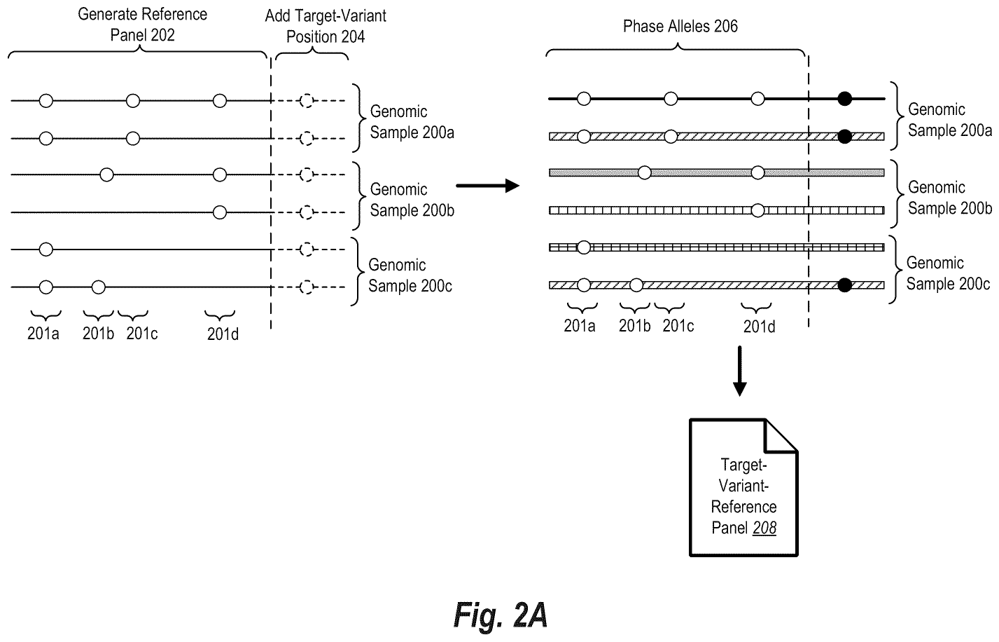

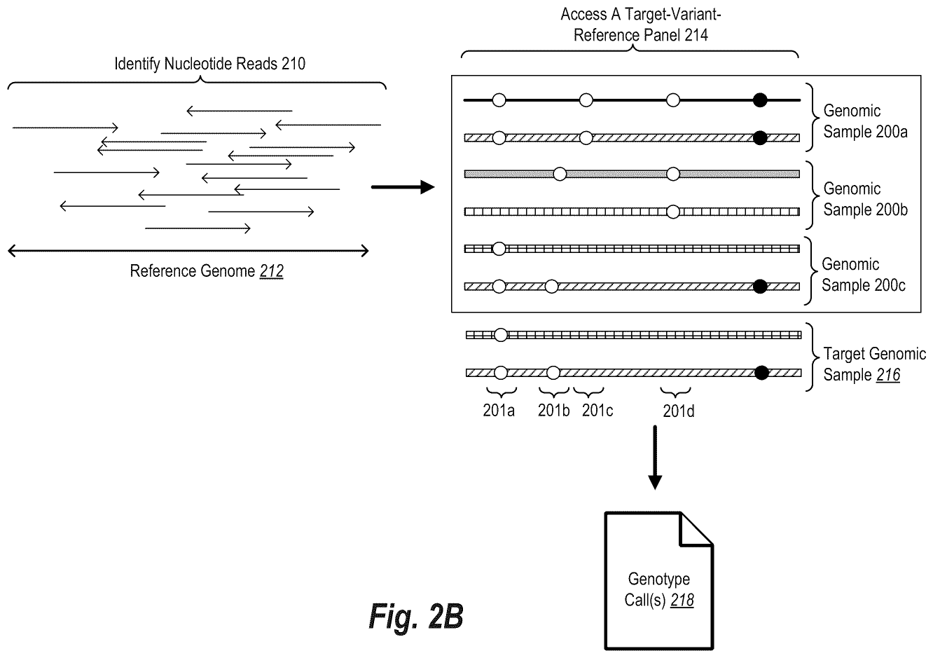

As mentioned above, in one or more embodiments, the customized genotype-imputation system 104 generates and/or utilizes a target-variant-reference panel to impute genotype calls. In accordance with one or more embodiments, FIG. 2A illustrates an overview of the customized genotype-imputation system 104 generating a target-variant-reference panel for a target variant, and FIG. 2B illustrates an overview of the customized genotype-imputation system 104 imputing genotype calls for the target variant utilizing the target-variant-reference panel.

As shown in FIG. 2A, for example, the customized genotype-imputation system 104 generates a reference panel 202. The reference panel 202 includes digital representations of haplotypes from genomic samples 200a, 200b, and 200c. Though FIG. 2A includes three genomic samples for purposes of illustration, it will be appreciated that, in one or more embodiments, the reference panel 202 can include a variety of quantities of diverse genomic samples.

As also shown in FIG. 2A, the customized genotype-imputation system 104 can generate the reference panel 202 to include phased alleles for the genomic samples 200a-200c. To illustrate, the customized genotype-imputation system 104 can determine which alleles from the genomic samples 200a-200c correspond to a maternal haplotype and a paternal haplotype. Accordingly, as shown in FIG. 2A, the reference panel 202 can include both maternal and paternal copies of each allele.

In addition to different haplotypes from the genomic samples 200a-200c, as further shown in FIG. 2A, the customized genotype-imputation system 104 generates the reference panel 202 including marker variants, such as SNPs and small indels (e.g., 10 or fewer base pairs, 50 or fewer base pairs). To mark individual marker variants in a corresponding genomic sample, the reference panel 202 includes marker-variant indicators 201a, 201b, 201c, and 201d at genomic coordinates for corresponding marker variants. In particular, FIG. 2A depicts open or unfilled circles within alleles of individual genomic samples of the genomic samples 200a-200c to represent marker-variant indicators 201a-201d. For purposes of illustration, an open or unfilled circle represents a marker-variant indicator that a particular allele of a genomic sample comprises a corresponding marker variant, and an absence of such an open or unfilled circle represents a marker-variant indicator that a particular allele of a genomic sample does not comprise the corresponding marker variant. Indeed, in one or more embodiments, the reference panel 202 includes data indications of other marker-variant indicators of marker variants present on either or both alleles corresponding to maternal or paternal haplotypes.

As also shown in FIG. 2A, the customized genotype-imputation system 104 adds a target-variant position 204 to the reference panel 202. More specifically, the customized genotype-imputation system 104 adds the target-variant position 204 to the reference panel 202 as part of generating a target-variant-reference panel. In one or more embodiments, the target-variant position 204 is a data field for indicating whether a target variant is present or absent for maternal and paternal alleles of a genomic sample. In particular, FIG. 2A depicts dotted-line circles alongside the alleles of the genomic samples 200a-200c to represent the target-variant position 204. Indeed, as shown in FIG. 2A, the customized genotype-imputation system 104 adds the target-variant position 204 for each genomic sample or for each allele of each genomic sample.

Having added the target-variant position 204, as further shown in FIG. 2A, the customized genotype-imputation system 104 phases alleles 206 of the genomic samples 200a-200c. More specifically, in one or more embodiments, the customized genotype-imputation system 104 phases allele(s) associated with the target variant to identify a genomic sequence comprising the target variant for either or both maternal and paternal alleles. Accordingly, the customized genotype-imputation system 104 can identify the presence or absence of the target variant for the maternal and paternal alleles of each genomic sample in the target-variant-reference panel. As depicted in FIG. 2A, for instance, the phased alleles 206 of the genomic samples 200a-200c include different patterns indicating different alleles corresponding to different haplotypes.

In addition to phasing alleles of different genomic samples, in one or more embodiments, the customized genotype-imputation system 104 adds target-variant indicators in the target-variant position 204. In particular, FIG. 2A depicts dark-filled circles alongside the alleles of the genomic samples 200a-200c to represent target-variant indicators within the target-variant position 204 to indicate the target variant is present within particular alleles. Indeed, the customized genotype-imputation system 104 generates target-variant indicators that indicate whether a genomic sample includes a target variant. Further, in one or more embodiments, the customized genotype-imputation system 104 adds the indicator to the target-variant position 204 for either of both maternal and paternal alleles of each genomic sample in a target-variant-reference panel.

By adding target-variant indicators to the target-variant position 204 and phasing alleles of the genomic samples 200a-200c, the customized genotype-imputation system 104 generates a target-variant-reference panel 208 including target-variant indicators in the target-variant position. Thus, the target-variant-reference panel 208 includes data for the target variant at each allele associated with the target variant. As shown in FIG. 2A, for instance, the target-variant-reference panel 208 is represented as a file. Indeed, in one or more embodiments, the customized genotype-imputation system 104 generates a VCF with the target-variant position 204 as a row in the VCF and the target-variant indicators as “0” for unaffected alleles and “1” for affected alleles.

Turning now to FIG. 2B, the customized genotype-imputation system 104 can utilize a target-variant-reference panel to impute genotype calls indicating a presence or absence of a target variant within a target genomic sample 216. To illustrate, as shown in FIG. 2B, the customized genotype-imputation system 104 identifies nucleotide reads 210 corresponding to the target genomic sample 216. In one or more embodiments, the customized genotype-imputation system 104 utilizes a sequencing system and/or one or more sequencing devices to identify nucleic-acid segments or oligonucleotides extracted from genomic samples to generate data. To illustrate, in some embodiments, a sequencing device or the customized genotype-imputation system 104 receives and analyzes, within nucleotide-sample slides (e.g., flow cells), oligonucleotides extracted from the target genomic sample 216. In addition, or in the alternative, the customized genotype-imputation system 104 can receive nucleotide reads for the target genomic sample 216 from a third-party sequencing system or from a sequencing device controlled by a separate entity.

As also shown in FIG. 2B, the customized genotype-imputation system 104 can align the nucleotide reads 210 with a reference genome 212 to determine variant calls or a sequence within certain genomic regions of the target genomic sample 216. Further, the customized genotype-imputation system 104 can identify one or more aligned nucleotide reads corresponding to a target genomic region for the target variant. Because alignment of the nucleotide reads 210 with the reference genome 212 may yield inaccurate variant calls or no calls for some genomic regions, the customized genotype-imputation system 104 can rely on a target-variant-reference panel 214 as an alternative to nucleotide reads covering a target genomic region. Accordingly, as shown in FIG. 2B, the customized genotype-imputation system 104 can further utilize the target-variant-reference panel 214 to determine genotype calls for the target genomic sample 216, especially for difficult-to-call genomic regions.

As shown in FIG. 2B, for example, the customized genotype-imputation system 104 accesses the target-variant-reference panel 214. To illustrate, in one or more embodiments, the customized genotype-imputation system 104 compares (i) marker variants that are exhibited by a subset of aligned nucleotide reads and that flank or surround a target genomic region for a target variant to (ii) corresponding marker variants within alleles of the genomic samples 200a-200c represented by the target-variant-reference panel 214. To illustrate such marker variants, the target-variant-reference panel 214 includes marker-variant indicators 201a, 201b, 201c, and 201d within alleles of the genomic samples 200a, 200b, and 200c. As noted above, an open or unfilled circle represents a marker-variant indicator that a particular allele of a genomic sample comprises a corresponding marker variant, and an absence of such an open or unfilled circle represents a marker-variant indicator that a particular allele of a genomic sample does not comprise the corresponding marker variant.

As further shown in FIG. 2B, both the genomic sample 200c and the target genomic sample 216 include open or unfilled circles representing the marker variant corresponding to the marker-variant indicator 201a on both maternal and paternal alleles. By contrast, both the genomic sample 200c and the target genomic sample 216 include a single open or unfilled circle representing the marker variant corresponding to the marker-variant indicator 201a on a single allele. The genomic sample 200c and the target genomic sample 216 otherwise include no such open or unfilled circle for the marker-variant indicators 201c and 201d to represent that their alleles do not comprise the corresponding marker variants.

To facilitate comparing marker variants among the target genomic sample 216 and the genomic samples 200a-200c represented by the target-variant-reference panel 214, the customized genotype-imputation system 104 can limit compared marker variants to a threshold distance from the target variant. Indeed, in one or more embodiments, the customized genotype-imputation system 104 identifies marker variants within a threshold number of nucleobases from the target variant or the target genomic region. For instance, in some cases, the customized genotype-imputation system 104 identifies marker variants (i) within a threshold number of nucleobases upstream of a target genomic region (e.g., 10, 50, 200 nucleobases) and/or (i) within a threshold number of nucleobases downstream from the target genomic region (e.g., 10, 50, 200 nucleobases).

Based on comparing such marker variants, the customized genotype-imputation system 104 can phase nucleotide reads of the target genomic sample 216 to identify corresponding alleles in maternal and paternal haplotypes. As indicated by the different patterns indicating different alleles in the target-variant-reference panel 214, for instance, the alleles of the target genomic sample 216 comprise the same marker variants as the alleles of the genomic sample 200c.

As further indicated by FIG. 2B, the customized genotype-imputation system 104 can impute genotype call(s) 218 for the target variant within the target genomic sample by comparing the marker variants of the target-variant-reference panel to the marker variants of the target genomic sample. More specifically, the customized genotype-imputation system 104 determines the genotype call(s) 218 by statistically inferring haplotypes likely to be present at the genomic region of the target genomic sample (e.g., represented as a value between 0 and 1) based on the target-variant-reference panel 214. To illustrate, the customized genotype-imputation system 104 utilizes statistical inference and haplotypes including marker variants from the target-variant-reference panel 214 to identify haplotypes from the target-variant-reference panel that are likely to be present at the genomic region. Further, the customized genotype-imputation system 104 can utilize the identified haplotypes from the target-variant-reference panel 214 to determine genotype calls for the target genomic sample.

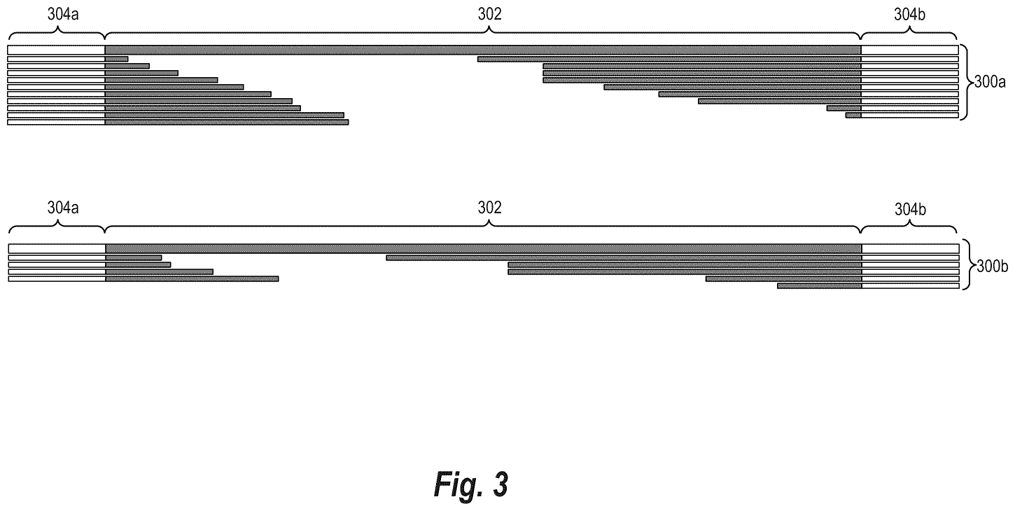

As mentioned above, many existing sequencing systems fail to make genotype calls or make inaccurate genotype calls for difficult-to-call genomic regions, including regions with repeat expansions. FIG. 3 illustrates such a difficult-to-call genomic region. More specifically, FIG. 3 illustrates nucleotide reads of a genomic sample misaligned with a genomic region comprising a repeat expansion in accordance with one or more embodiments.

As shown by FIG. 3, for instance, a sequencing system aligns nucleotide reads corresponding to a genomic sample 300a (e.g., HG04127) and a genomic sample 300b (e.g., HG01506) with (i) a target genomic region 302 and of a reference genome corresponding to a repeat expansion for the RFC1 gene and (ii) surrounding genomic regions 304a and 304b adjacent to the target genomic region 302. Both the genomic samples 300a and 300b are putative carriers of a repeat-expansion variant corresponding to the target genomic region 302. As shown, the sequencing system aligns nucleotide reads of the genomic sample 300a with at least 10 times coverage for the genomic sample 300a with the surrounding genomic regions 304a and 304b, but inconsistently aligns nucleotides reads of the genomic sample 300a with the target genomic region 302. Similarly, the sequencing system aligns nucleotide reads of the genomic sample 300b with at least 4 times coverage for the genomic sample 300b with the surrounding genomic regions 304a and 304b, but inconsistently aligns nucleotides reads of the genomic sample 300b with the target genomic region 302. Despite both the genomic sample 300a and the genomic sample 300b being putative carriers of the repeat-expansion variant, the alignment exhibits read-coverage holes within the target genomic region 302.

FIG. 3 accordingly illustrates poor nucleotide-read data quality that is characteristic of some genomic regions that exhibit repeat expansions. In some cases, such expansion repeats are impossible to accurately identify in genomic samples that carry the target variant utilizing existing sequencing systems. More specifically, the alignment of the nucleotide reads with the reference genome in the target genomic region 302 is uncertain or impossible due to the nature of the repeat giving a variety of possible alignments. For example, as shown in FIG. 3, the genomic samples 300a and 300b respectively exhibit approximately 35 and 33 repeat units of AAGGG within the target genomic region 302. Because a nucleotide fragment indicating “AGGGAAGGGAAG,” for instance, could have a variety of alignments, existing sequencing systems find it difficult or even impossible to align corresponding nucleotide reads with the target genomic region 302 of the reference genome and determine the length of the repeat expansion.

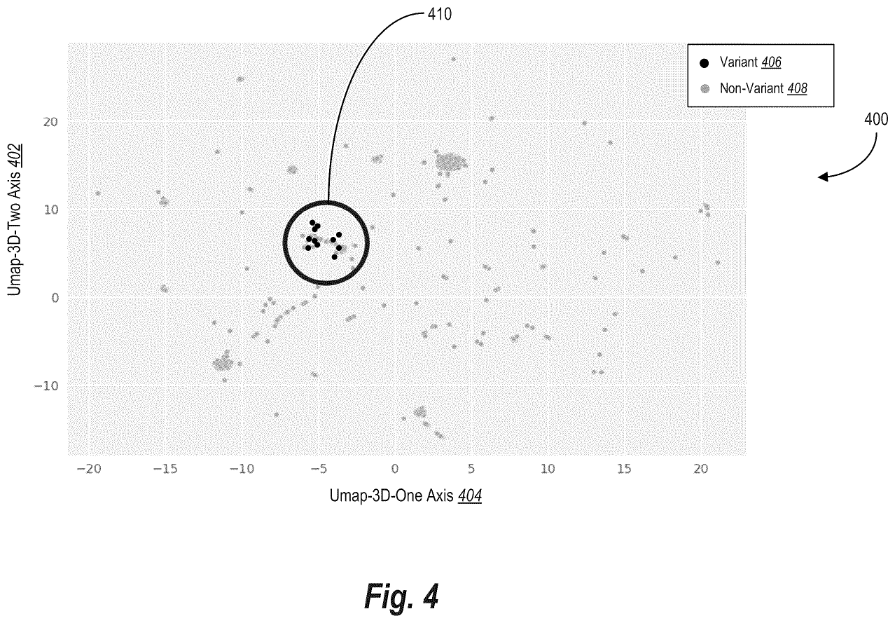

As mentioned above, the customized genotype-imputation system 104 can utilize a target-variant-reference panel to impute more accurate genotype calls for target variants than existing sequencing systems, especially for difficult-to-call genomic regions. In accordance with one or more embodiments, FIG. 4 illustrates a Uniform Manifold Approximation and Projection (UMAP) graph 400 of data points representing various genomic samples clustered according to SNPs or other marker variants. As indicated by a target-variant cluster 410 in the UMAP graph 400, genomic samples affected by a target variant tend to cluster together based on shared marker variants.

As indicated by FIG. 4, in one or more embodiments, the customized genotype-imputation system 104 performs Principal Component Analysis (PCA) to cluster genomic samples based on SNPs or other marker variants present in each genomic sample. The customized genotype-imputation system 104 further utilizes UMAP to visualize clusters of the genomic samples. The UMAP graph 400 exhibits the results of such clustering.

As depicted in FIG. 4, for instance, the UMAP graph 400 shows data points representing various genomic samples along a UMAP-3D-One axis 404 and a UMAP-3D-Two axis 402 via dimension reduction. As shown by dark-filled circles indicating certain data points, the UMAP graph 400 includes data points representing genomic samples carrying a variant 406 for the RFC1 gene that includes a pathogenic repeat expansion. In particular, the customized genotype-imputation system 104 identifies the target-variant cluster 410 comprising data points representing genomic samples that include at least one allele with the target variant of the RFC1 gene. By contrast, as shown by the lighter-filled circles or grey circles representing certain data points, the UMAP graph 400 also includes data points representing genomic samples exhibiting a non-variant 408 or, in other words, genomic samples that are not affected by the target variant of the RFC1 gene.

Accordingly, the UMAP graph 400 shows that SNPs or other marker variants constitute reliable evidence for imputation of genotype calls for the target variant of RFC1. To illustrate, the genomic samples from the target-variant cluster 410 because they not only exhibit the same or similar nucleotides at the target genomic region for RFC1, but also similar or the same SNPs at other genomic regions flanking or surrounding the target genomic region (e.g., within 200 base pairs upstream or downstream from the target genomic region). Therefore, the UMAP graph 400 demonstrates a proof of concept that SNPs can be used to infer or identify genomic samples that exhibit RFC1 pathogenic repeats.

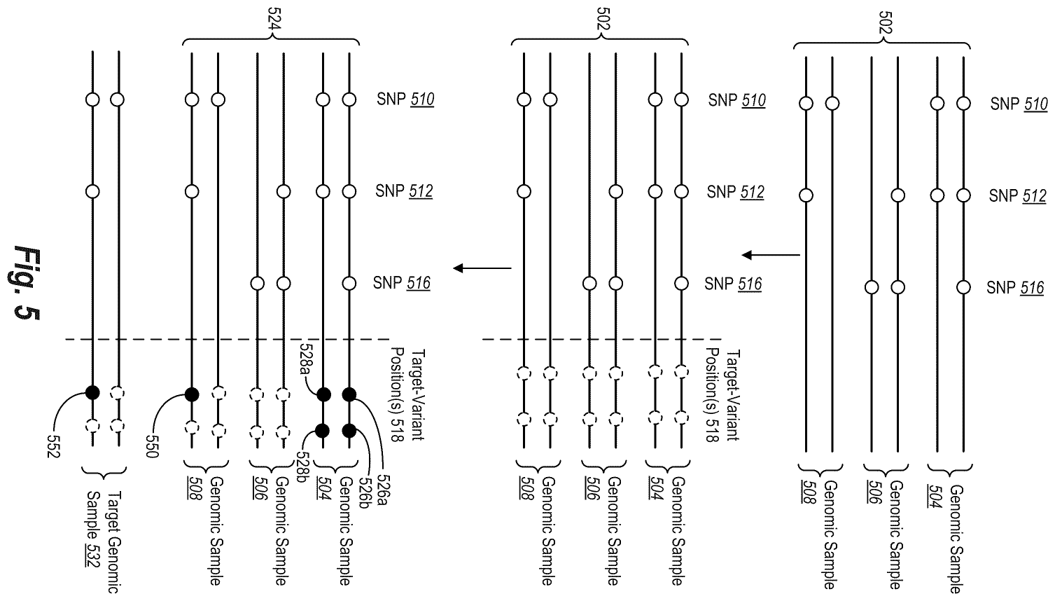

To leverage such a concept using a unique reference panel specific to a target variant, the customized genotype-imputation system 104 can generate a target-variant-reference panel including a target-variant position. In accordance with one or more embodiments, FIG. 5 illustrates the customized genotype-imputation system 104 generating a reference panel 502 and adding target-variant position(s) 518 to the reference panel 502 to generate a target-variant-reference panel 524. As explained below, the customized genotype-imputation system 104 can generate the target-variant-reference panel 524 including (i) phased alleles corresponding to target-variant indicators within the target-variant position(s) 518 and (ii) marker-variant indicators for marker variants phased according to the maternal haplotypes and the paternal haplotypes of genomic samples.

As shown in FIG. 5, the customized genotype-imputation system 104 generates the reference panel 502 including genomic samples 504, 506, and 508 of different haplotypes. In particular, the reference panel 502 includes alleles of the genomic samples 504-508 comprising marker-variant indicators for SNPs 510, 512, and 516. However, it will be appreciated that the genomic samples 504-508 and the SNPs 510-516 are given by way of example, and that the customized genotype-imputation system 104 can generate reference panels and/or target-variant-reference panels including a variety of quantities of SNPs and genomic samples, including genomic samples representing hundreds or thousands of haplotypes and thousands of SNPs (e.g., 50,000; 100,000 SNPs).

As indicated above, in one or more embodiments, the customized genotype-imputation system 104 generates the reference panel 502 including genomic samples with a variety of different haplotypes exhibiting genetic diversity. To illustrate, the customized genotype-imputation system 104 can generate the reference panel 502 including the genomic samples 504-508 from a variety of ancestries, continents, countries, and/or populations. Likewise, the customized genotype-imputation system 104 can transform the reference panel 502 into a target-variant-reference panel that includes the genomic samples 504-508 with marker variants from a variety of different ancestries, continents, countries, and/or populations.

As indicated above, in one or more embodiments, the customized genotype-imputation system 104 can generate an output file (e.g., VCF) comprising data representing the reference panel 502 and/or the target-variant-reference panel 524. For illustrative purposes, however, FIG. 5 depicts the reference panel 502 and the target-variant-reference panel 524 as collections of lines representing haplotypes of the genomic samples 504-508 and circles representing marker-variant indicators indicating the presence of the SNPs 510-512. As indicated by the open or empty circles representing marker-variant indicators, the genomic sample 504 includes the SNP 510 for both maternal and paternal alleles, the SNP 512 for both maternal and paternal alleles, and one copy of the SNP 516. By contrast, the genomic sample 506 includes one copy of the SNP 512 and includes the SNP 516 on both maternal and paternal alleles. As also shown in FIG. 5, the genomic sample 508 includes the SNP 510 on both maternal and paternal alleles and includes one copy of the SNP 512.

While FIG. 5 illustrates marker-variant indicators for the SNPs as open or empty circles, it will be appreciated that in one or more embodiments, the reference panel 502 and/or the target-variant-reference panel 524 can be represented within an output file (e.g., VCF) including data fields with “0” reflecting a reference nucleobase and “1” reflecting an alternate nucleobase. In addition, or in the alternative, the customized genotype-imputation system 104 can utilize alternate binary schemes for marker-variant indicators. For example, the customized genotype-imputation system 104 can generate the reference panel 502 and/or the target-variant-reference panel 524 including two cells or positions for a multi-allelic marker variant, where a “0” as a maker-variant indicator in both positions reflects a reference nucleobase, a “0” and “1” as maker-variant indicators in the first and second positions reflect a first alternate nucleobase, a “1” and “1” as maker-variant indicators in the first and second positions reflect a second alternate nucleobase, and a “1” and “0” as maker-variant indicators in the first and second reflect a third alternate nucleobase. Alternatively, as a further example, in some embodiments, the customized genotype-imputation system 104 can generate the reference panel 502 and/or the target-variant-reference panel 524 including a single cell or position for a multi-allelic marker variant, where the value “0” reflects a reference nucleobase, “1” reflects a first alternate nucleobase, “2” reflects a second alternate nucleobase, and/or “3” reflects a third alternate nucleobase.

As further shown in FIG. 5, the customized genotype-imputation system 104 utilizes the SNPs 510-516 as marker variants for imputation of genotype calls for a presence or absence of a target variant within a target genomic sample. However, in one or more embodiments, the customized genotype-imputation system 104 can utilize other marker variants, such as a marker variant in the form of a deletion, an insertion, a duplication, an inversion, a translocation, or a CNV. In some cases, the customized genotype-imputation system 104 can generate the reference panel 502 comprising data fields with values (e.g., a sequence of values) identifying a variety of marker variant types.

As shown in FIG. 5, the customized genotype-imputation system 104 generates the target-variant-reference panel 524 in part by adding the target-variant position(s) 518. As mentioned briefly above, the target-variant position(s) 518 can correspond to a variety of target variants. For example, the target variant can include biallelic or multi-allelic variants. Further, in one or more embodiments, the target variant includes a repeat expansion, such as an STR expansion or a VNTR expansion. Regardless of whether the target variant constitutes a repeat expansion, in some cases, the target variant constitutes a pathogenic variant.

More specifically, in one or more embodiments, the target variant can include a variant of various genes. To illustrate, in some embodiments, the target variant can include, but is not limited to, a variant of a Replication Factor C Subunit 1 (RFC1) gene, a Cytochrome P450 Family 2 Subfamily D Member 6 (CYP2D6) gene, Cytochrome P450 Family 2 Subfamily B Member 6 (CYP2B6) gene, Cytochrome P450 Family 21 Subfamily A Member 2 (CYP21A2) gene, Survival Motor Neuron 1 (SMN1) gene, Survival Motor Neuron 2 (SMN2) gene, Glucosylceramides Beta (GBA) gene, Blood Group Rh(CE) (RHCE) gene, Lipoprotein(A) (LPA) gene, a Fragile X Mental Retardation 1 (FMR1) gene, a Hexosaminidase Subunit Alpha (HEXA) gene, Hemoglobin Subunit Alpha 1 (HBA1) gene, Hemoglobin Subunit Alpha 2 (HBA2) gene, or a Hemoglobin Subunit Beta (HBB) gene.

Regardless of the gene or target genomic region, in some embodiments, the target variant can include a deletion, an insertion, a duplication, an inversion, a translocation, or a CNV transmitted within a population. To illustrate, in one or more embodiments, the customized genotype-imputation system 104 uses a target variant that is inherited from an ancestral haplotype to support data sufficient for a target-variant-reference panel with a target-variant position specific to the target variant. Accordingly, in some embodiments, de novo variants may not support a target-variant-reference panel. Because the customized genotype-imputation system 104 detects variants based on a target-variant-reference panel including various genomic samples, a new mutation in the target genomic sample would not be present in a sufficient number of haplotypes to support a functional version of the target-variant-reference panel. Thus, the new variant would not be present or only have limited representation in the target-variant-reference panel.

To ensure sufficient haplotype data, in one or more embodiments, the customized genotype-imputation system 104 uses a target-variant-reference panel specific to a target variant that satisfies one or more thresholds. For example, in some cases, the target variant must satisfy one or more relative thresholds depending on a number of genomic samples in the target-variant-reference panel—including a threshold carrier frequency, a threshold linkage disequilibrium (LD) with respect to particular marker variants, or a threshold mutation rate. To support imputing genotype calls, in one or more embodiments with a target-variant-reference panel representing approximately 3,000 genomic samples, the target variant must exhibit a threshold carrier frequency of approximately 2% of genomic samples; a threshold LD at r² of 0.75 with SNPs or other marker variants, thereby mimicking a strong founder effect; and a threshold mutation rate of 1.29×10⁻⁸ mutations per base pair per meiosis.

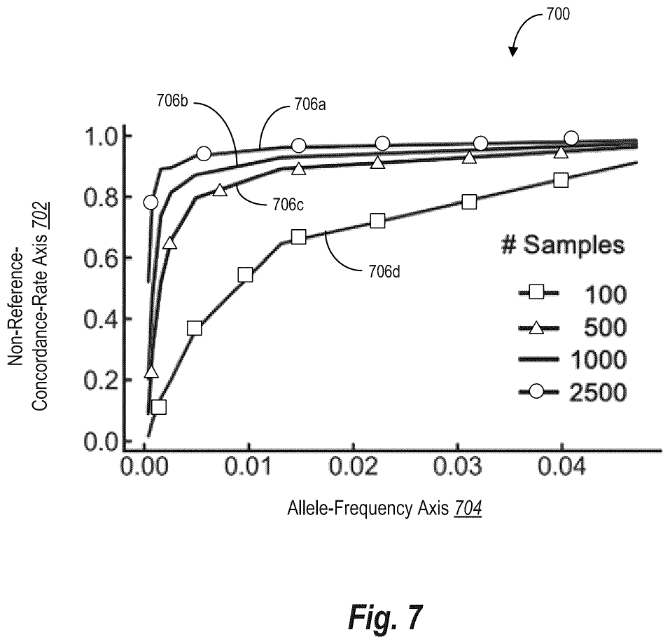

Indeed, in some embodiments, the customized genotype-imputation system 104 determines the threshold carrier frequency, the threshold linkage disequilibrium, or the threshold mutation rate relative to the number of genomic samples represented by the target-variant-reference panel. For instance, a target-variant-reference panel representing a relatively larger number of genomic samples may facilitate a relatively lower threshold carrier frequency, relatively lower threshold linkage disequilibrium, or a relatively lower threshold mutation rate. Accordingly, other suitable measures may be used for a threshold carrier frequency, a threshold LD, or a threshold mutation rate than the examples provided above. As described below, FIG. 7 provides examples of different threshold carrier frequencies for a target variant depending on a number of genomic samples represented by a target-variant-reference panel.

As further shown in FIG. 5, in one or more embodiments, the customized genotype-imputation system 104 adds the target-variant position(s) 518 to the reference panel 502 by adding one or more data fields associated with a target variant. As mentioned above, the customized genotype-imputation system 104 can generate the target-variant-reference panel 524 as a VCF file and can utilize various binary schemes to indicate nucleotides at genomic coordinates. To illustrate, in some embodiments, each target-variant position(s) 518 can be a field comprising a target-variant indicator of either a “0” or “1,” where “0” represents a reference nucleobase and “1” represents an alternate nucleobase.

By using various different target-variant indicators, the customized genotype-imputation system 104 can generate a target-variant-reference panel for biallelic or multi-allelic target variants. By using two fields for two target-variant positions, for example, the customized genotype-imputation system 104 can represent multi-allelic target variants. Indeed, as shown in FIG. 5, a couple of dotted-lined circles in each allele of the genomic samples 504, 506, and 508 represent the target-variant position(s) 518 as two target-variant positions (e.g., data fields) that together can include or facilitate binary code indicating a presence or absence a multi-allelic target variant for a given genomic sample.

To illustrate how such a binary code in two target-variant positions indicates a multi-allelic target variant, in some embodiments, a “0” as a target-variant indicator in both target-variant positions represents a reference nucleobase (e.g., A). By contrast, a “0” as a target-variant indicator in a first target-variant position and a “1” as a target-variant indicator in a second target-variant position represents a first alternate nucleobase (e.g., G). Further, a “1” as a target-variant indicator in a first target-variant position and a “1” as a target-variant indicator in a second target-variant position represents a second alternate nucleobase (e.g., T). A “1” as a target-variant indicator in a first target-variant position and a “0” as a target-variant indicator in a second target-variant position represents a third alternate nucleobase (e.g., C).

In the alternative to multiple target-variant positions, in some embodiments, the customized genotype-imputation system 104 uses a non-binary code in a single target-variant position to indicate a presence or absence of a multi-allelic target variant. Although not represented by FIG. 5, in some embodiments, a “0” as a target-variant indicator in the target-variant position represents a reference nucleobase (e.g., A), a “1” as a target-variant indicator in the target-variant position represents a first alternate nucleobase (e.g., G), a “2” as a target-variant indicator in the target-variant position represents a second alternate nucleobase (e.g., T), and a “3” as a target-variant indicator in the target-variant position represents a first alternate nucleobase (e.g., G).

As shown in FIG. 5, for example, the target-variant-reference panel 524 includes target-variant indicators 526a and 526b as dark-filled circles on one allele and 528a and 528b as dark-filled circles on another allele to indicate that genomic sample 504 includes a particular haplotype of a multi-allelic target variant on both maternal and paternal alleles. Conversely, the target-variant-reference panel 524 includes a couple of dotted-lined circles on both alleles of the genomic sample 506 to indicate that the genomic sample 506 does not include the multi-allelic target variant on either the maternal or paternal alleles. Further, the target-variant-reference panel 524 includes a target-variant indicator 550 as a dark-filled circle on an allele of the genomic sample 508 to indicate that the genomic sample 508 includes one copy of the multi-allelic target variant on either maternal or paternal allele, and a dotted-lined circle on one allele of the genomic sample 508 to indicate that the genomic sample 508 does not include the multi-allelic target variant on one allele.

As further indicated by FIG. 5, in addition to adding the target-variant position(s) 518, in some embodiments, the customized genotype-imputation system 104 phases alleles of the genomic samples 504-508 together with target-variant indicators in the target-variant position for the target variant. By phasing the alleles of the genomic samples 504-508, the customized genotype-imputation system 104 determines a presence or absence of the target variant in corresponding alleles present on maternal haplotypes and paternal haplotypes of the genomic samples 504-508. To phase such alleles, in some cases, the customized genotype-imputation system 104 executes a haplotype phasing model, such as Segmented HAPlotype Estimation and Imputation Tool (SHAPEIT), to estimate haplotypes from the genotype data corresponding to the genomic samples 504-508.

Because both alleles of a homozygous genomic sample include a copy of a target variant and corresponding target-variant indicators in a target-variant-reference panel, in some embodiments, the customized genotype-imputation system 104 phases heterozygous alleles of a subset of genomic samples, such as the genomic sample 508, where the alleles are heterozygous for the target variant. Indeed, in some cases, the customized genotype-imputation system 104 does not phase homozygous alleles of a subset of genomic samples, such as the genomic samples 504 and 506. By contrast, in some embodiments, the customized genotype-imputation system 104 executes a haplotype phasing model to phase alleles of genomic samples represented by the target-variant-reference panel 524 regardless of the genomic sample's zygosity for a target variant, where the data representing the alleles being phased in the target-variant-reference panel also includes target-variant indicators in the target-variant position for the target variant.

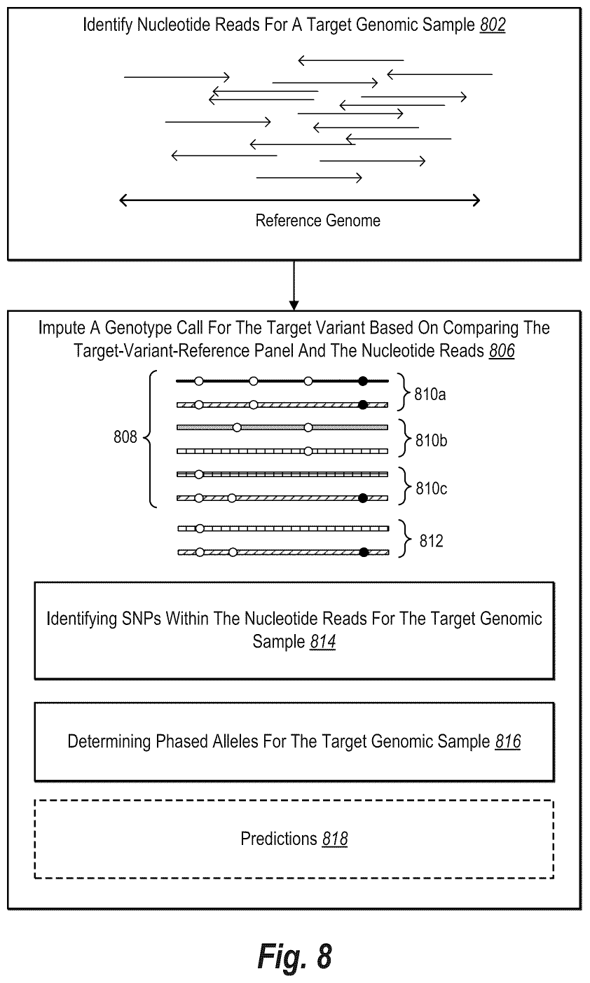

As further indicated by FIG. 5, the customized genotype-imputation system 104 can compare nucleotide reads of a target genomic sample 532 to the target-variant-reference panel 524. As will be discussed below with regard to FIG. 8, the customized genotype-imputation system 104 can impute genotype calls for a target variant within a target genomic sample utilizing the target-variant-reference panel 524. More specifically, the customized genotype-imputation system 104 can utilize the target-variant-reference panel 524 to determine phased genotype calls for both maternal and paternal copies from the target genomic sample 532.

As indicated by FIG. 5, the customized genotype-imputation system 104 generates a genotype call that the target genomic sample 532 comprises the multi-allelic target variant and exhibits the same haplotype as the genomic sample 508. Indeed, similar to the genomic sample 508, the target genomic sample 532 includes a target-variant indicator 552 as a dark-filled circle on an allele to indicate that the target genomic sample 532 includes one copy of the multi-allelic target variant on either maternal or paternal allele, and a dotted-lined circle on one allele to indicate that the target genomic sample 532 does not include the multi-allelic target variant on one allele.

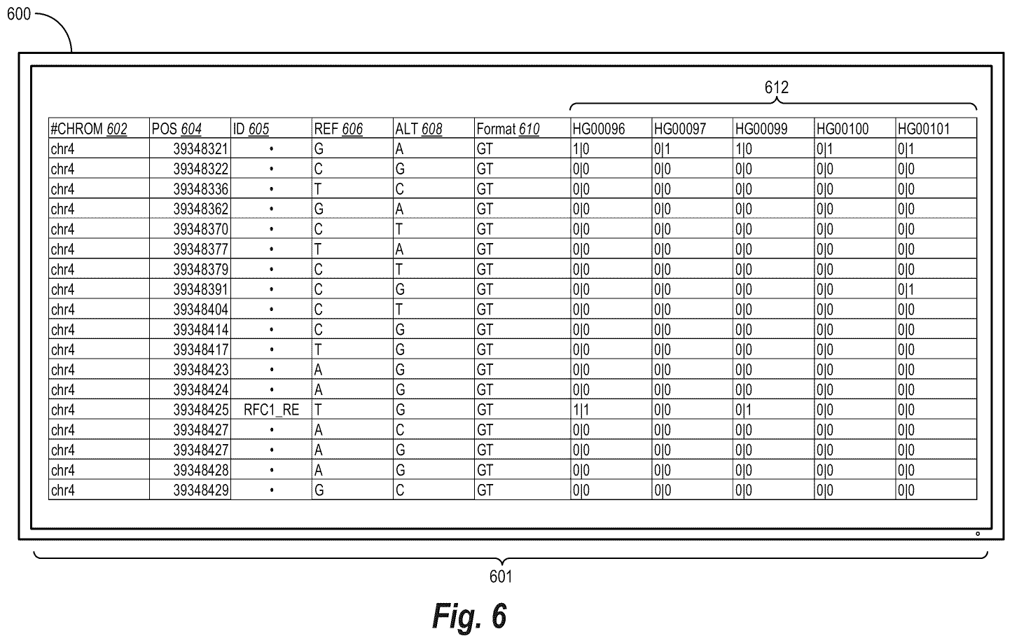

As mentioned above, the customized genotype-imputation system 104 can generate an output file comprising a target-variant-reference panel. In accordance with one or more embodiments, FIG. 6 illustrates a client device 600 presenting a portion of an example VCF comprising a target-variant-reference panel 601 within a graphical user interface. As explained below, the target-variant-reference panel 601 includes indicators of nucleobase calls at genomic coordinates for a variety of genomic samples and target-variant indicators in target-variant positions indicating whether a particular allele of a genomic sample exhibits a target variant.

As shown in FIG. 6, for example, the target-variant-reference panel 601 includes a chromosome column 602, a coordinate column 604, a target-variant column 605, a reference-nucleobase column 606, an alternate-nucleobase column 608, a format column 610, and genomic-sample columns 612. Though FIG. 6 illustrates the client device 600 presenting a portion of the target-variant-reference panel 601, it will be appreciated that the target-variant-reference panel 601 can include information concerning alleles across an entire genome, and that the provided genomic coordinates are merely illustrative.

As further shown in FIG. 6, the chromosome column 602 includes chromosome information for each row. To illustrate, in FIG. 6, the client device 600 presents rows for nucleobase calls for genomic coordinates on chromosome 4. Additionally, the coordinate column 604 includes partial genomic coordinates for each row indicating which genomic coordinate corresponds to the nucleobase-call information in that row. In particular, as shown in the graphical user interface depicted in FIG. 6, the client device 600 presents genomic coordinates from chr4:39348321 to chr4:39348429.

Additionally, the client device 600 presents information for a reference nucleobase (e.g., a non-variant nucleotide base) in the reference-nucleobase column 606, such as a single-letter code (e.g., A, C, T, G) in each cell representing the reference base from the reference genome at the corresponding genomic coordinate. Further, the client device 600 presents information for an alternate nucleobase (e.g., a variant nucleotide base) in the alternate-nucleobase column 608, such as a single-letter code (e.g., A, C, T, G) in each cell representing a most common alternate nucleobase or a called alternate nucleobase at the corresponding genomic coordinate.

As further shown in FIG. 6, the client device 600 also presents information for a format of a nucleobase call provided in the format column 610 and values for phased nucleobase calls in the genomic-sample columns 612 for particular genomic samples. As shown in FIG. 6, the target-variant-reference panel 601 includes the text “GT” indicating a genotype-call format fort allele values of “0” or “1” in the genomic-sample columns 612. More specifically, a value of “0” indicates that the nucleobase call is the reference nucleobase from the reference-nucleobase column 606. By contrast, a value of “1” indicates that the nucleobase call is the alternate nucleobase from the alternate-nucleobase column 608. The “1” symbol in between values in the genomic-sample columns 612 indicates a phased genotype call.

In addition to genotype calls for marker variants and other genomic coordinates, the client device 600 also presents a target-variant column 605 that includes an identifier for a target variant. As shown in FIG. 6, the target-variant-reference panel 601 includes an identifier for RCF1 at the genomic coordinate chr4:39348425. In some embodiments, chr4:39348425 represents a placeholder genomic coordinate for the target-variant position rather than an actual genomic coordinate within a reference genome. Indeed, the row corresponding to chr4:3934825 includes a cell or a field that represents an example target-variant position for each of genomic samples HG00096, HG00097, HG00099, HG00100, and HG00101.

In particular, as shown in the target-variant-reference panel 601, the row corresponding to chr4:3934825 includes “0” and “1” values as target-variant indicators for a presence or absence of the target variant within genomic samples HG00096, HG00097, HG00099, HG00100, and HG00101. By separating the “0” and “1” values with “1” as a symbol for a straight bar, the target-variant-reference panel 601 includes phased target-variant indicators for maternal and paternal alleles of each respective genomic sample. Accordingly, the client device 600 provides information regarding target variants in the target-variant-reference panel 601 via the graphical user interface.

As part of improving genotype-calling accuracy for target variants, in some embodiments, the customized genotype-imputation system 104 can use a target-variant-reference panel representing different numbers of genomic samples. In accordance with one or more embodiments, FIG. 7 illustrates a graph 700 that plots non-reference-concordance rates at which a sequencing system accurately imputes target variants of varying allele frequencies based on target-variant-reference panels representing different numbers of genomic samples. As indicated by FIG. 7, the non-reference-concordance-rate curves show that the customized genotype-imputation system 104 more accurately imputes genotype calls for target variants as the genomic-sample size of the target-variant-reference panel increases. The graph 700 further illustrates how the customized genotype-imputation system 104 can use non-reference-concordance rates and allele frequencies to determine a threshold carrier frequency depending on a genomic-sample size of a target-variant-reference panel.

To test the accuracy of imputation for different reference panels, for example, researchers removed certain target variants from data representing target genomic samples sequenced by a sequencing device. The customized genotype-imputation system 104 subsequently imputed genotype calls for the target variants from the target genomic samples based on corresponding target-variant-reference panels of varying genomic-sample size. As indicated by FIG. 7, a first target-variant-reference panel corresponding to non-reference-concordance-rate curve 706d includes about 100 genomic samples; a second target-variant-reference panel corresponding to non-reference-concordance-rate curve 706c includes about 500 genomic samples; a third target-variant-reference panel corresponding to non-reference-concordance-rate curve 706b includes about 1,000 genomic samples; and a fourth target-variant-reference panel corresponding to non-reference-concordance-rate curve 706a includes about 2,500 genomic samples.

As shown in the graph 700, the graph 700 includes values for non-reference-concordance rates along a non-reference-concordance-rate axis 702 and values for allele frequency along an allele-frequency axis 704. In particular, the non-reference-concordance-rate axis 702 represents an accuracy of genotype-call imputation in terms of a non-reference-concordance rate from 0 to 1.0 (e.g., where 0 represents no concordance and 1.0 represents total concordance). In the graph 700, the value of such a non-reference-concordance rate represents a quotient of (i) a true positive rate at which a sequencing system imputes target variants over (ii) a sum of a false positive rate, the true positive rate, and a false negative rate at which the sequencing system imputes target variants, which can be represented as TPR/FPR+TPR+FNR. Further, the allele-frequency axis 704 represents the allele frequency (also called carrier frequency) for a target variant from 0.00 to 0.05.

According to the non-reference-concordance-rate axis 702 and the allele-frequency axis 704 of the graph 700, the customized genotype-imputation system 104 improves an accuracy of genotype-call imputation for target variants as the number of genomic samples represented by a target-variant-reference panel increases. In particular, the non-reference-concordance-rate curve 706d for the customized genotype-imputation system 104 using the first target-variant-reference panel representing 100 genomic samples indicates a lowest non-reference-concordance rate for imputing the removed target variants across allele frequencies for the target variants. By contrast, the non-reference-concordance-rate curve 706a for the customized genotype-imputation system 104 using the fourth target-variant-reference panel representing 2,500 genomic samples indicates a highest non-reference-concordance rate for imputing the removed target variants across allele frequencies for the target variants. Indeed for each of the non-reference-concordance-rate curves 706a, 706b, and 706c, the non-reference-concordance rate increases with the allele frequency before plateauing at maximum concordance at an allele frequency at around 0.02.

Accordingly, in some embodiments, the customized genotype-imputation system 104 can accurately impute genotype calls for target variants exhibiting at least a 2% threshold carrier frequency by using a target-variant-reference panel representing 500 or more genomic samples. Indeed, as shown by the non-reference-concordance-rate curve 706a, the customized genotype-imputation system 104 can accurately impute genotype calls for relatively less common target variants (e.g., with a carrier frequency of 2% or less) by using a target-variant-reference panel comprising 2,500 genomic samples. Further, in some embodiments, the customized genotype-imputation system 104 can accurately impute genotype calls for target variants exhibiting at least a 5% threshold carrier frequency by using a target-variant-reference panel representing about 100 or more genomic samples. Indeed, as shown by the non-reference-concordance-rate curve 706d, the customized genotype-imputation system 104 can accurately impute genotype calls for relatively more common target variants (e.g., with a carrier frequency of 5% or less) with a target-variant-reference panel representing 100 genomic samples.

As mentioned above, the customized genotype-imputation system 104 can also utilize a target-variant-reference panel. In accordance with one or more embodiments, FIG. 8 illustrates the customized genotype-imputation system 104 imputing genotype calls indicating a presence or absence of a target variant within a target genomic sample utilizing a target-variant-reference panel. As an overview, the customized genotype-imputation system 104 (i) identifies nucleotide reads for a target genomic sample, (ii) accesses a target-variant-reference panel comprising target-variant indicators within a target-variant position for phased alleles of genomic samples of different haplotypes, and (iii) imputes a genotype call for the target variant within the target genomic sample based on comparing alleles of the haplotypes represented by the target-variant-reference panel to the nucleotide reads for the target genomic sample.

As shown in FIG. 8, for example, the customized genotype-imputation system 104 performs an act 802 of identifying nucleotide reads for a target genomic sample. In some cases, for instance, the customized genotype-imputation system 104 receives data representing nucleotide reads for a genomic sample that have been sequenced by a sequencing device. Such data for the nucleotide reads includes a sequence of nucleobase calls determined by the sequencing device. After receiving the read data, the customized genotype-imputation system 104 can align the nucleotide reads with a reference genome. Based on the aligned nucleotide reads, the customized genotype-imputation system 104 can determine one or more nucleobase calls for genomic coordinates and genomic regions of the target genomic sample with respect to the reference genome.

As further shown in FIG. 8, the customized genotype-imputation system 104 performs an act 806 of imputing a genotype call for the target variant based on comparing the target-variant-reference panel and the nucleotide reads. To illustrate, in one or more embodiments, the customized genotype-imputation system 104 accesses a target-variant-reference panel 808, such as by accessing a VCF stored locally or on one or more client devices within the computing system 100. In one or more embodiments, the customized genotype-imputation system 104 provides and/or receives the target-variant-reference panel 808 over a network.

As shown in FIG. 8, the target-variant-reference panel 808 includes dark-filled circles representing target-variant indicators within a target-variant position for phased alleles of genomic samples 810a, 810b, and 810c of different haplotypes. The target-variant-reference panel 808 also includes empty or open circles representing marker-variant indicators for marker variants within the phased alleles of the genomic samples 810a-810c. As depicted, the phased alleles of the genomic samples 810a-810c include different patterns indicating different alleles corresponding to different haplotypes. Similarly, FIG. 8 depicts the alleles of a target genomic sample 812 to include various patterns to represent different alleles.

Based on a comparison of (i) a subset of nucleotide reads of the target genomic sample 812 corresponding to a target genomic region for a target variant with (ii) alleles of the genomic samples 810a-810c within the target-variant-reference panel 808, the customized genotype-imputation system 104 imputes genotype call(s) for the target genomic sample 812. More specifically, in some embodiments, the customized genotype-imputation system 104 imputes genotype calls corresponding to genomic coordinates of the target genomic region based on marker variants surrounding or flanking the target genomic region for the target variant.

As shown in FIG. 8, in one or more embodiments, the act 806 further includes an act 814 of identifying SNPs within the nucleotide reads for the target genomic sample. More specifically, in one or more embodiments, the customized genotype-imputation system 104 compares marker variants surrounding the target genomic region on the target genomic sample 812 with marker variants on the genomic samples 810a-810c included in the target-variant-reference panel 808. Indeed, in one or more embodiments, the customized genotype-imputation system 104 identifies marker variants within a threshold number of nucleobases from the target variant. For instance, in some cases, the customized genotype-imputation system 104 identifies marker variants within a threshold number of nucleobases (e.g., 50 base pairs, 200 base pairs, 500 base pairs) upstream of a target genomic region and/or within a threshold number of nucleobases (e.g., 50 base pairs, 200 base pairs, 500 base pairs) downstream from the target genomic region. As indicated above, FIG. 8 depicts marker-variant indicators for marker variants (e.g., SNPs) as empty or open circles within the phased alleles of the genomic samples 810a-810c and the target genomic sample 812.

To illustrate a comparison of marker variants, the customized genotype-imputation system 104 can determine SNPs in the genomic coordinates surrounding or flanking the target genomic region on the target genomic sample 812 and the SNPs in the genomic coordinates surrounding or flanking the target genomic region on the genomic samples 810a-810c in the target-variant-reference panel 808. Based on the SNPs (or other marker variants) common between the haplotypes of the target genomic sample 812 and haplotypes of the genomic samples 810a-810c in the target-variant-reference panel 808, the customized genotype-imputation system 104 statistically infers which nucleobases or which alleles are more likely present within the target genomic region on the target genomic sample 812.

As also shown in FIG. 8, in some embodiments, the act 806 of imputing a genotype call includes an act 816 of determining phased alleles for the target genomic sample 812. To illustrate, in one or more embodiments, the customized genotype-imputation system 104 phases the nucleotide reads of the target genomic sample 812 based on the marker variants (e.g., SNPs) in the nucleotide reads of the target genomic sample 812 and marker variants in the genomic samples 810a-810c. By comparing marker variants and phasing the nucleotide reads with respect to haplotypes in the target-variant-reference panel 808, the customized genotype-imputation system 104 identifies alleles of the target genomic sample 812 in the target genomic region also present in the maternal and paternal haplotypes of the genomic samples 810a-810c.

As indicated by the different patterns indicating different alleles in the target-variant-reference panel 808, for instance, the alleles of the target genomic sample 812 comprise the same marker variants as the alleles of the genomic sample 810c. Because the customized genotype-imputation system 104 can identify shared alleles between the target genomic sample 812 and one or more haplotypes of the genomic samples 810a-810c—and identify target-variant indicators within a target-variant position for the genomic samples 810a-810c of the target-variant-reference panel 808—the customized genotype-imputation system 104 can generate phased genotype calls indicating a presence or absence of a target variant on particular alleles within the target genomic sample 812. As indicating by the dark-filled circles representing target-variant indicators in the target-variant-reference panel 808, the customized genotype-imputation system 104 can statistically infer that a particular allele of the target genomic sample 812 includes a target variant because a corresponding allele of the genomic sample 810c includes a target-variant indicator in a target-variant position. Indeed, by applying a haplotype phasing model and a genotype imputation model to the target-variant-reference panel 808, the customized genotype-imputation system 104 can determine a phased genotype call indicating the presence or absence of the target variant at an allele of the target genomic sample 812 corresponding to a maternal or paternal haplotype represented in the target-variant-reference panel 808.

As just indicated, in one or more embodiments, the customized genotype-imputation system 104 utilizes a haplotype phasing model to phase the nucleotide reads from the target genomic sample 812. In one or more embodiments, the customized genotype-imputation system 104 utilizes Segmented HAPlotype Estimation and Imputation Tool (SHAPEIT) to estimate haplotypes from the genotype data, including the nucleotide reads of the target genomic sample 812 and genomic sequences of the genomic samples 810a-810c in the target-variant-reference panel 808. To illustrate, in one or more embodiments, the customized genotype-imputation system 104 utilizes the SHAPEIT algorithm to perform a Positional Burrow Wheeler Transformation (PBWT) to efficiently select a set of relevant haplotypes to be used to phase nucleotide reads of the target genomic sample 812. Accordingly, the customized genotype-imputation system 104 can pre-process and extract phase information from the set of relevant haplotypes. In one or more embodiments, the customized genotype-imputation system 104 can also utilize a haplotype scaffold or parental haplotype data to phase the nucleotide reads of the target genomic sample 812. Thus, the customized genotype-imputation system 104 can utilize the phase information from the set of relevant haplotypes and, optionally, haplotype scaffold or parental haplotype data to write a VCF or BCF file phasing the target genomic sample 812. In one or more embodiments, the customized genotype-imputation system 104 utilizes HTSlib to write the VCF or BCF file.

In some embodiments, for instance, the customized genotype-imputation system 104 uses SHAPEIT to phase haplotypes as described by Olivier Delaneau, Jean-Francois Zagury et al., Scalable and Integrative Haplotype Estimation, Nat. Comm. (2019), which is hereby incorporated by reference in its entirety.

As also mentioned above, in one or more embodiments, the customized genotype-imputation system 104 applies a genotype imputation model, such as a hidden Markov model (HMM)-based genotype imputation model, to impute genotype calls for the target region corresponding to the target variant. To illustrate, in some embodiments, the customized genotype-imputation system 104 can identify relevant haplotypes from the genomic samples 810a-810c in the target-variant-reference panel 808 utilizing an HMM-based genotype imputation model. More specifically, the customized genotype-imputation system 104 can utilize an HMM-based genotype imputation model to (i) compare marker variants corresponding to the target genomic region of the target genomic sample 812 and marker variants in the haplotypes of the target genomic region within the genomic samples 810a-810c and (ii) identify likely haplotypes corresponding to the target genomic region present in the target genomic sample 812.

In one or more embodiments, the customized genotype-imputation system 104 utilizes Genotype Likelihoods Imputation and PhaSing mEthod (GLIMPSE) as a genotype imputation model, as described by Simone Rubinacci et al., “Efficient Phasing and Imputation of Low-coverage Sequencing Data Using Large Reference Panels,” 53 Nature Genetics 120-126 (2021), which is hereby incorporated by reference in its entirety. More specifically, in some embodiments, the customized genotype-imputation system 104 utilizes GLIMPSE to determine posterior genotype likelihoods for the target genomic region corresponding to the target variant for the target genomic sample 812. Indeed, in some embodiments, the customized genotype-imputation system 104 executes SHAPEIT to phase nucleotide reads from a target genomic sample before executing GLIMPSE to impute genotype calls for a target variant based on a target-variant-reference panel.

As mentioned above, in one or more embodiments, the customized genotype-imputation system 104 generates a target-variant-reference panel including one or more target genomic regions (or genomic regions of interest) corresponding to a target variant and excluding other genomic coordinates or genomic regions. To illustrate, in some embodiments, the customized genotype-imputation system 104 limits a target-variant-reference panel to include data representing haplotypes of genomic samples corresponding to one or more target genomic regions corresponding to a target variant, but not data representing haplotypes outside the one or more target genomic regions. Indeed, in one or more embodiments, the customized genotype-imputation system 104 includes data representing haplotypes from genomic samples for multiple target genomic regions, including different chromosomes, in a target-variant-reference panel corresponding to multiple target variants. For example, the customized genotype-imputation system 104 can generate a target-variant-reference panel comprising data representing different haplotypes corresponding to a target variant for the CYP2D6 gene at a target genomic region (e.g., chr4:35149660-47004037). In some cases, the same target-variant-reference panel comprises data representing different haplotypes corresponding to an additional target variant for the RFC1 gene at an additional target genomic region (e.g., chr22: 37149660-54004037).

Indeed, in one or more embodiments, the customized genotype-imputation system 104 inputs data for such a target-variant-reference panel for only target genomic regions into a genotype imputation model (e.g., GLIMPSE). By reducing or eliminating unnecessary genomic regions and using a target-variant-reference panel comprising data limited to one or more target genomic regions, the customized genotype-imputation system 104 uses less memory to store the target-variant-reference panel and expedites the computer-processing time for executing a genotype imputation model to impute genotype calls for target variants.

In the alternative to GLIMPSE, in some embodiments, the customized genotype-imputation system 104 uses a different HMM-based genotype imputation model to impute haplotypes, such as the model described by Genetic Variants Predictive of Cancer Risk, WO 2013/035/114 A1 (published Mar. 14, 2013), or by A. Kong et al., Detection of Sharing by Descent, Long-Range Phasing and Haplotype Imputation, Nat. Genet. 40, 1068-75 (2008), both of which are incorporated by reference in their entirety. Additionally, or alternatively, the customized genotype-imputation system 104 uses other available software, such as BEAGLE, MACH, or IMPUTE, to impute genotype calls.

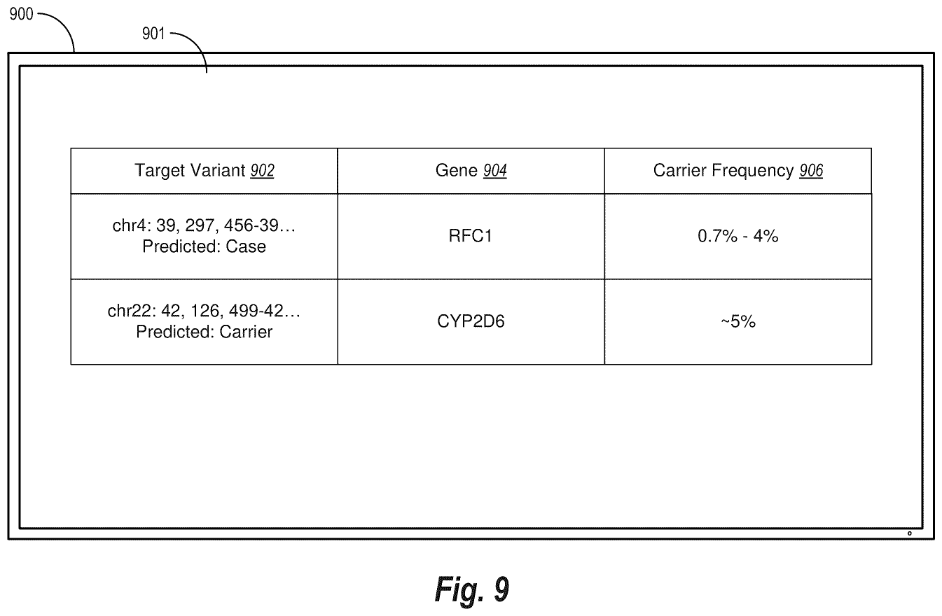

As further shown in FIG. 8, the customized genotype-imputation system 104 can optionally perform an act 818 of generating predictions of whether target genomic samples comprise a target variant. To illustrate, in one or more embodiments, the customized genotype-imputation system 104 can utilize the determined genotype calls to generate predictions of whether a target genomic sample comprises a pathogenic variant at an allele present on a maternal haplotype or a paternal haplotype. As discussed below with regard to FIG. 9, the customized genotype-imputation system 104 can provide such predictions to a client device via a graphical user interface.

In some embodiments, for instance, the customized genotype-imputation system 104 can utilize an inheritance pattern associated with a condition or disease corresponding to the target variant to generate predictions. To illustrate, the customized genotype-imputation system 104 can determine whether a condition associated with the target variant is autosomal recessive, autosomal dominant, X-linked, Y-linked, codominant, or a variety of inheritance patterns. More specifically, the customized genotype-imputation system 104 compares the inheritance pattern to the genotype calls to generate the predictions. In some embodiments, the predictions indicate whether a target genomic sample is a carrier of a target variant at a particular allele, a case of the target variant at both alleles, or unaffected by the target variant at either allele.

After determining imputed genotype calls, in one or more embodiments, the customized genotype-imputation system 104 provides information concerning such imputed genotype calls for one or more target variants via a graphical user interface. In accordance with one or more embodiments, FIG. 9 illustrates a client device 900 presenting a graphical user interface 901 comprising information concerning imputed genotype calls for target variants. While FIG. 9 depicts the graphical user interface 901 displayed when the client device 900 implements computer-executable instructions of the customized genotype-imputation system 104, rather than repeatedly refer to the computer-executable instructions causing the client device 900 to perform certain actions for the customized genotype-imputation system 104, this disclosure describes the client device 900 or the customized genotype-imputation system 104 performing those actions in the following paragraphs.

As shown in FIG. 9, for instance, the client device 900 provides data in a target-variant column 902, a gene column 904, and a carrier frequency column 906. To illustrate, the target-variant column 902 includes data identifying a target variant and a corresponding prediction. More specifically, client device 900 presents genomic coordinates for a target variant and a prediction as to whether a target genomic sample includes the target variant (e.g., pathogenic variant). To illustrate, in one or more embodiments, the customized genotype-imputation system 104 provides the client device 900 with a prediction of whether a pathogenic variant is present in the target genomic sample at an allele on one or both of a maternal haplotype and a paternal haplotype.

Based on the imputed genotype call, therefore, the client device 900 can present a prediction as to whether a target genomic sample is affected by one or more target variants. As shown in FIG. 9, for instance, the client device 900 presents the target-variant column 902 including “Predicted: Case” for a first target variant within the target genomic sample at genomic coordinates “chr4: 39, 287, 456-39 . . . ” As indicated by “Predicted: Case,” the customized genotype-imputation system 104 predicts that the target genomic sample comprises the first target variant of the RFC1 gene on both alleles. Accordingly, in some cases, the prediction indicates a potential phenotype of the target genomic sample on the Cerebellar Ataxia, Neuropathy, Vestibular Areflexia Syndrome (CANVAS) spectrum. As further shown in FIG. 9, the client device 900 presents the target-variant column 902 including “Predicted: Carrier” for a second target variant within the target genomic sample at genomic coordinates “chr22: 42, 126, 499-42 . . . . ” As indicated by “Predicted: Carrier,” in some cases, the customized genotype-imputation system 104 predicts that the target genomic sample comprises the second target variant of the CYP2D6 gene on one allele. Accordingly, the prediction indicates that the target genomic sample carries a variant of a genetic indicator for Neuroleptic Malignant Syndrome.

As further shown in FIG. 9, the client device 900 presents annotations for a gene and a carrier frequency corresponding to the target variants and their corresponding predictions. For instance, the client device 900 presents the gene column 904 including “RFC1” and “CYP2D6” corresponding to the predictions in the target-variant column 902 for the first target variant and the second target variant, respectively. In addition to specific gene identification, the client device 900 presents carrier frequencies in the carrier frequency column 906. More specifically, the client device 900 presents a carrier frequency of 0.7%-4% for the first target variant on the RFC1 gene and −5% for the second target variant on the CYP2D6 gene. In some embodiments, the carrier frequency represents the frequencies of the target variants from a genomic sample database or from metadata corresponding to the target-variant-reference panel. By providing the predictions, genomic coordinates, genes, and carrier frequencies, the customized genotype-imputation system 104 provides clinicians, test subjects, or other people with critical information indicating variant calls for certain genes.

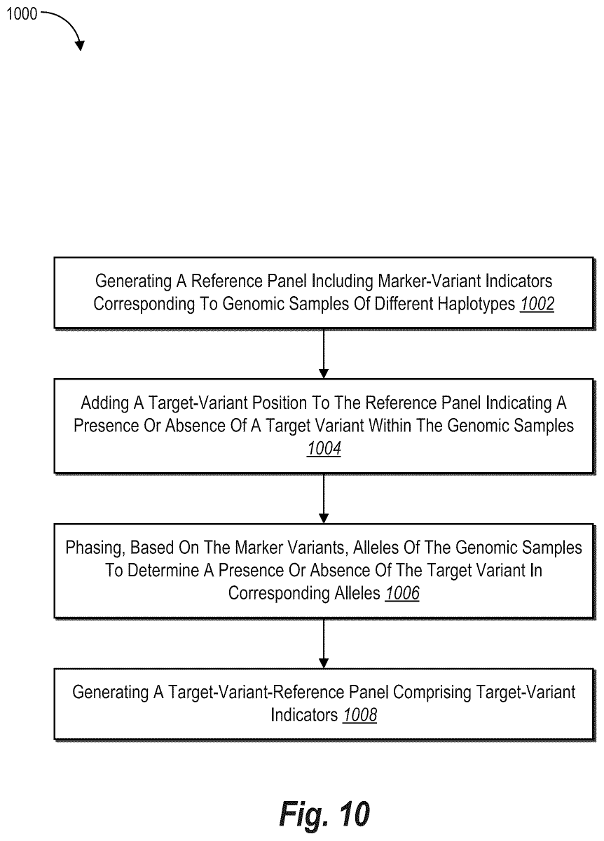

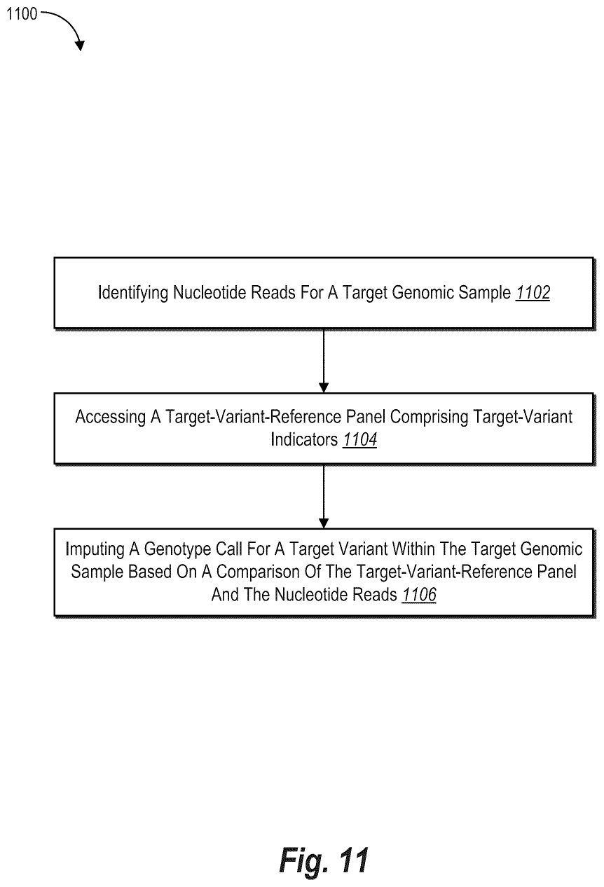

FIGS. 1-9, the corresponding text, and the examples provide a number of different methods, systems, devices, and non-transitory computer-readable media of the customized genotype-imputation system 104. In addition to the foregoing, one or more embodiments can also be described in terms of flowcharts comprising acts for accomplishing a particular result, as shown in FIGS. 10-11. FIGS. 10-11 may be performed with more or fewer acts. Further, the acts may be performed in differing orders. Additionally, the acts described herein may be repeated or performed in parallel with one another or parallel with different instances of the same or similar acts.

As mentioned, FIG. 10 illustrates a flowchart of a series of acts 1000 for generating a target-variant-reference panel in accordance with one or more embodiments. While FIG. 10 illustrates acts according to one embodiment, alternative embodiments may omit, add to, reorder, and/or modify any of the acts shown in FIG. 10. The acts of FIG. 10 can be performed as part of a method. Alternatively, a non-transitory computer-readable medium can comprise instructions that, when executed by one or more processors, cause a computing device or a system to perform the acts of FIG. 10. In some embodiments, a system can perform the acts of FIG. 10.

As shown in FIG. 10, the series of acts 1000 includes an act 1002 for generating a reference panel including marker-variant indicators corresponding to genomic samples of different haplotypes. In particular, the act 1002 can include generating a reference panel comprising marker-variant indicators for marker variants at genomic coordinates corresponding to genomic samples of different haplotypes. Specifically, in some cases, the at least one target-variant position comprises a target-variant position for target-variant indicators of a biallelic target variant. Additionally, in one or more embodiments, in some cases, the at least one target-variant position comprises multiple target-variant positions for target-variant indicators of a multi-allelic target variant.

Further, in one or more embodiments, in some cases, the marker variants comprise single-nucleotide polymorphisms (SNPs).

As shown in FIG. 10, the series of acts 1000 includes an act 1004 for adding a target-variant position to the reference panel indicating a presence or absence of a target variant within the genomic samples. In particular, the act 1004 can include adding at least one target-variant position to the reference panel indicating a presence or absence of a target variant within the genomic samples. Specifically, in some cases, the target variant comprises a repeat expansion. Additionally, in one or more embodiments, the act 1004 includes wherein the target variant comprises a deletion, an insertion, a duplication, an inversion, a translocation, or a copy number variation (CNV) transmitted within a population. The act 1004 can also include wherein the target variant satisfies one or more of a threshold carrier frequency, a threshold linkage disequilibrium (LD) with respect to particular marker variants, or a threshold mutation rate.

Further, in one or more embodiments, the target variant comprises a variant of a Replication Factor C Subunit 1 (RFC1) gene, a Cytochrome P450 Family 2 Subfamily D Member 6 (CYP2D6) gene, Cytochrome P450 Family 2 Subfamily B Member 6 (CYP2B6) gene, Cytochrome P450 Family 21 Subfamily A Member 2 (CYP21A2) gene, Survival Motor Neuron 1 (SMN1) gene, Survival Motor Neuron 2 (SMN2) gene, Glucosylceramides Beta (GBA) gene, Blood Group Rh(CE) (RHCE) gene, Lipoprotein(A) (LPA) gene, a Fragile X Mental Retardation 1 (FMR1) gene, a Hexosaminidase Subunit Alpha (HEXA) gene, Hemoglobin Subunit Alpha 1 (HBA1) gene, Hemoglobin Subunit Alpha 2 (HBA2) gene, or a Hemoglobin Subunit Beta (HBB) gene.

As shown in FIG. 10, the series of acts 1000 includes an act 1006 for phasing, based on the marker variants, alleles of the genomic samples to determine a presence or absence of the target variant in corresponding alleles. In particular, the act 1006 can include phasing, based on the marker variants, alleles of the genomic samples to determine a presence or absence of the target variant in corresponding alleles present on maternal haplotypes and paternal haplotypes. Specifically, in some cases, phasing the alleles of the genomic samples comprises phasing heterozygous alleles of a subset of the genomic samples.

As shown in FIG. 10, the series of acts 1000 includes an act 1008 for generating a target-variant-reference panel comprising target-variant indicators. In particular, the act 1008 can include generating a target-variant-reference panel comprising target-variant indicators within the at least one target-variant position for the phased alleles of the genomic samples. Specifically, in some cases, generating the reference panel comprises generating a phased reference panel comprising the marker-variant indicators for marker variants phased according to the maternal haplotypes and the paternal haplotypes of the genomic samples. Additionally, in one or more embodiments, in some cases, the genomic samples of different haplotypes comprise genomic samples of different haplotypes exhibiting genetic diversity. In some cases, the target-variant-reference panel comprises marker-variant indicators for marker variants within a target genomic region for the target variant and does not comprise additional marker-variant indicators for additional marker variants outside of the target genomic region.

Additionally, FIG. 11 illustrates a flowchart of a series of acts 1100 for utilizing a target-variant-reference panel to impute genotype calls in accordance with one or more embodiments. While FIG. 11 illustrates acts according to one embodiment, alternative embodiments may omit, add to, reorder, and/or modify any of the acts shown in FIG. 11. The acts of FIG. 11 can be performed as part of a method. Alternatively, a non-transitory computer-readable medium can comprise instructions that, when executed by one or more processors, cause a computing device or a system to perform the acts of FIG. 11. In some embodiments, a system can perform the acts of FIG. 11.

As shown in FIG. 11, the series of acts 1100 includes an act 1102 for identifying nucleotide reads for a target genomic sample. In particular, the act 1102 can include identifying nucleotide reads corresponding to a target genomic sample.

As shown in FIG. 11, the series of acts 1100 includes an act 1104 for accessing a target-variant-reference panel comprising target-variant indicators. In particular, the act 1104 can include accessing a target-variant-reference panel comprising target-variant indicators within at least one target-variant position for phased alleles of genomic samples of different haplotypes. Specifically, in some cases, the target-variant indicators indicate a presence or absence of the target variant in the at least one target-variant position for the phased alleles of the genomic samples. In some cases, the target-variant-reference panel comprises marker-variant indicators for marker variants within a target genomic region for the target variant and does not comprise additional marker-variant indicators for additional marker variants outside of the target genomic region.

As shown in FIG. 11, the series of acts 1100 includes an act 1106 for imputing a genotype call for a target variant within the target genomic sample based on a comparison of the target-variant-reference panel and the nucleotide reads. In particular, the act 1106 can include impute a genotype call for a target variant within the target genomic sample based on a comparison of the target-variant-reference panel and the nucleotide reads corresponding to the target genomic sample. Specifically, the act 1106 can include determining phased alleles of the target genomic sample based on the comparison of the target-variant-reference panel and the nucleotide reads corresponding to the target genomic sample, and imputing the genotype call by imputing a phased genotype call for the target variant within the target genomic sample based on the phased alleles of the target genomic sample.

Additionally, in one or more embodiments, the act 1106 includes imputing the genotype call for the target variant by generating a prediction of whether the target genomic sample comprises the target variant. Further, in some embodiments, generating the prediction comprises predicting whether the target genomic sample comprises a pathogenic variant at an allele present on a maternal haplotype or a paternal haplotype.

The act 1106 can also include imputing the genotype call by identifying, within the nucleotide reads corresponding to the target genomic sample, one or more single-nucleotide polymorphisms (SNPs) as one or more marker variants within the target-variant-reference panel for the target variant, and determining the genotype call further based on the one or more SNPs within the nucleotide reads. Further, the act 1106 can include impute the genotype call for the target variant by imputing the genotype call for a repeat expansion. Additionally, the act 1106 can include imputing the genotype call utilizing a genotype imputation model.

The methods described herein can be used in conjunction with a variety of nucleic acid sequencing techniques. Particularly applicable techniques are those wherein nucleic acids are attached at fixed locations in an array such that their relative positions do not change and wherein the array is repeatedly imaged. Embodiments in which images are obtained in different color channels, for example, coinciding with different labels used to distinguish one nucleotide base type from another are particularly applicable. In some embodiments, the process to determine the nucleotide sequence of a target nucleic acid (i.e., a nucleic-acid polymer) can be an automated process. Preferred embodiments include sequencing-by-synthesis (SBS) techniques.

SBS techniques generally involve the enzymatic extension of a nascent nucleic acid strand through the iterative addition of nucleotides against a template strand. In traditional methods of SBS, a single nucleotide monomer may be provided to a target nucleotide in the presence of a polymerase in each delivery. However, in the methods described herein, more than one type of nucleotide monomer can be provided to a target nucleic acid in the presence of a polymerase in a delivery.

SBS can utilize nucleotide monomers that have a terminator moiety or those that lack any terminator moieties. Methods utilizing nucleotide monomers lacking terminators include, for example, pyrosequencing and sequencing using 7-phosphate-labeled nucleotides, as set forth in further detail below. In methods using nucleotide monomers lacking terminators, the number of nucleotides added in each cycle is generally variable and dependent upon the template sequence and the mode of nucleotide delivery. For SBS techniques that utilize nucleotide monomers having a terminator moiety, the terminator can be effectively irreversible under the sequencing conditions used as is the case for traditional Sanger sequencing which utilizes dideoxynucleotides, or the terminator can be reversible as is the case for sequencing methods developed by Solexa (now Illumina, Inc.).

SBS techniques can utilize nucleotide monomers that have a label moiety or those that lack a label moiety. Accordingly, incorporation events can be detected based on a characteristic of the label, such as fluorescence of the label; a characteristic of the nucleotide monomer such as molecular weight or charge; a byproduct of incorporation of the nucleotide, such as release of pyrophosphate; or the like. In embodiments, where two or more different nucleotides are present in a sequencing reagent, the different nucleotides can be distinguishable from each other, or alternatively, the two or more different labels can be the indistinguishable under the detection techniques being used. For example, the different nucleotides present in a sequencing reagent can have different labels and they can be distinguished using appropriate optics as exemplified by the sequencing methods developed by Solexa (now Illumina, Inc.).

Preferred embodiments include pyrosequencing techniques. Pyrosequencing detects the release of inorganic pyrophosphate (PPi) as particular nucleotides are incorporated into the nascent strand (Ronaghi, M., Karamohamed, S., Pettersson, B., Uhlen, M. and Nyren, P. (1996) “Real-time DNA sequencing using detection of pyrophosphate release.” Analytical Biochemistry 242(1), 84-9; Ronaghi, M. (2001) “Pyrosequencing sheds light on DNA sequencing.” Genome Res. 11(1), 3-11; Ronaghi, M., Uhlen, M. and Nyren, P. (1998) “A sequencing method based on real-time pyrophosphate.” Science 281(5375), 363; U.S. Pat. Nos. 6,210,991; 6,258,568 and 6,274,320, the disclosures of which are incorporated herein by reference in their entireties). In pyrosequencing, released PPi can be detected by being immediately converted to adenosine triphosphate (ATP) by ATP sulfurylase, and the level of ATP generated is detected via luciferase-produced photons. The nucleic acids to be sequenced can be attached to features in an array and the array can be imaged to capture the chemiluminescent signals that are produced due to incorporation of a nucleotides at the features of the array. An image can be obtained after the array is treated with a particular nucleotide type (e.g., A, T, C or G). Images obtained after addition of each nucleotide type will differ with regard to which features in the array are detected. These differences in the image reflect the different sequence content of the features on the array. However, the relative locations of each feature will remain unchanged in the images. The images can be stored, processed and analyzed using the methods set forth herein. For example, images obtained after treatment of the array with each different nucleotide type can be handled in the same way as exemplified herein for images obtained from different detection channels for reversible terminator-based sequencing methods.

In another exemplary type of SBS, cycle sequencing is accomplished by stepwise addition of reversible terminator nucleotides containing, for example, a cleavable or photobleachable dye label as described, for example, in WO 04/018497 and U.S. Pat. No. 7,057,026, the disclosures of which are incorporated herein by reference. This approach is being commercialized by Solexa (now Illumina Inc.), and is also described in WO 91/06678 and WO 07/123,844, each of which is incorporated herein by reference. The availability of fluorescently-labeled terminators in which both the termination can be reversed, and the fluorescent label cleaved facilitates efficient cyclic reversible termination (CRT) sequencing. Polymerases can also be co-engineered to efficiently incorporate and extend from these modified nucleotides.

Preferably in reversible terminator-based sequencing embodiments, the labels do not substantially inhibit extension under SBS reaction conditions. However, the detection labels can be removable, for example, by cleavage or degradation. Images can be captured following incorporation of labels into arrayed nucleic acid features. In particular embodiments, each cycle involves simultaneous delivery of four different nucleotide types to the array and each nucleotide type has a spectrally distinct label. Four images can then be obtained, each using a detection channel that is selective for one of the four different labels. Alternatively, different nucleotide types can be added sequentially and an image of the array can be obtained between each addition step. In such embodiments, each image will show nucleic acid features that have incorporated nucleotides of a particular type. Different features are present or absent in the different images due the different sequence content of each feature. However, the relative position of the features will remain unchanged in the images. Images obtained from such reversible terminator-SBS methods can be stored, processed and analyzed as set forth herein. Following the image capture step, labels can be removed and reversible terminator moieties can be removed for subsequent cycles of nucleotide addition and detection. Removal of the labels after they have been detected in a particular cycle and prior to a subsequent cycle can provide the advantage of reducing background signal and crosstalk between cycles. Examples of useful labels and removal methods are set forth below.

In particular embodiments some or all of the nucleotide monomers can include reversible terminators. In such embodiments, reversible terminators/cleavable fluors can include fluor linked to the ribose moiety via a 3′ ester linkage (Metzker, Genome Res. 15:1767-1776 (2005), which is incorporated herein by reference). Other approaches have separated the terminator chemistry from the cleavage of the fluorescence label (Ruparel et al., Proc Natl Acad Sci USA 102: 5932-7 (2005), which is incorporated herein by reference in its entirety). Ruparel et al described the development of reversible terminators that used a small 3′ allyl group to block extension, but could easily be deblocked by a short treatment with a palladium catalyst. The fluorophore was attached to the base via a photocleavable linker that could easily be cleaved by a 30 second exposure to long wavelength UV light. Thus, either disulfide reduction or photocleavage can be used as a cleavable linker. Another approach to reversible termination is the use of natural termination that ensues after placement of a bulky dye on a dNTP. The presence of a charged bulky dye on the dNTP can act as an effective terminator through steric and/or electrostatic hindrance. The presence of one incorporation event prevents further incorporations unless the dye is removed. Cleavage of the dye removes the fluor and effectively reverses the termination. Examples of modified nucleotides are also described in U.S. Pat. Nos. 7,427,673, and 7,057,026, the disclosures of which are incorporated herein by reference in their entireties.

Additional exemplary SBS systems and methods which can be utilized with the methods and systems described herein are described in U.S. Patent Application Publication No. 2007/0166705, U.S. Patent Application Publication No. 2006/0188901, U.S. Pat. No. 7,057,026, U.S. Patent Application Publication No. 2006/0240439, U.S. Patent Application Publication No. 2006/0281109, PCT Publication No. WO 05/065814, U.S. Patent Application Publication No. 2005/0100900, PCT Publication No. WO 06/064199, PCT Publication No. WO 07/010,251, U.S. Patent Application Publication No. 2012/0270305 and U.S. Patent Application Publication No. 2013/0260372, the disclosures of which are incorporated herein by reference in their entireties.

Some embodiments can utilize detection of four different nucleotides using fewer than four different labels. For example, SBS can be performed utilizing methods and systems described in the incorporated materials of U.S. Patent Application Publication No. 2013/0079232. As a first example, a pair of nucleotide types can be detected at the same wavelength, but distinguished based on a difference in intensity for one member of the pair compared to the other, or based on a change to one member of the pair (e.g., via chemical modification, photochemical modification or physical modification) that causes apparent signal to appear or disappear compared to the signal detected for the other member of the pair. As a second example, three of four different nucleotide types can be detected under particular conditions while a fourth nucleotide type lacks a label that is detectable under those conditions, or is minimally detected under those conditions (e.g., minimal detection due to background fluorescence, etc.). Incorporation of the first three nucleotide types into a nucleic acid can be determined based on presence of their respective signals and incorporation of the fourth nucleotide type into the nucleic acid can be determined based on absence or minimal detection of any signal. As a third example, one nucleotide type can include label(s) that are detected in two different channels, whereas other nucleotide types are detected in no more than one of the channels. The aforementioned three exemplary configurations are not considered mutually exclusive and can be used in various combinations. An exemplary embodiment that combines all three examples, is a fluorescent-based SBS method that uses a first nucleotide type that is detected in a first channel (e.g. dATP having a label that is detected in the first channel when excited by a first excitation wavelength), a second nucleotide type that is detected in a second channel (e.g. dCTP having a label that is detected in the second channel when excited by a second excitation wavelength), a third nucleotide type that is detected in both the first and the second channel (e.g. dTTP having at least one label that is detected in both channels when excited by the first and/or second excitation wavelength) and a fourth nucleotide type that lacks a label that is not, or minimally, detected in either channel (e.g. dGTP having no label).

Further, as described in the incorporated materials of U.S. Patent Application Publication No. 2013/0079232, sequencing data can be obtained using a single channel. In such so-called one-dye sequencing approaches, the first nucleotide type is labeled but the label is removed after the first image is generated, and the second nucleotide type is labeled only after a first image is generated. The third nucleotide type retains its label in both the first and second images, and the fourth nucleotide type remains unlabeled in both images.

Some embodiments can utilize sequencing by ligation techniques. Such techniques utilize DNA ligase to incorporate oligonucleotides and identify the incorporation of such oligonucleotides. The oligonucleotides typically have different labels that are correlated with the identity of a particular nucleotide in a sequence to which the oligonucleotides hybridize. As with other SBS methods, images can be obtained following treatment of an array of nucleic acid features with the labeled sequencing reagents. Each image will show nucleic acid features that have incorporated labels of a particular type. Different features are present or absent in the different images due the different sequence content of each feature, but the relative position of the features will remain unchanged in the images. Images obtained from ligation-based sequencing methods can be stored, processed and analyzed as set forth herein. Exemplary SBS systems and methods which can be utilized with the methods and systems described herein are described in U.S. Pat. No. 6,1069,488, U.S. Pat. Nos. 6,172,218, and 6,306,597, the disclosures of which are incorporated herein by reference in their entireties.

Some embodiments can utilize nanopore sequencing (Deamer, D. W. & Akeson, M. “Nanopores and nucleic acids: prospects for ultrarapid sequencing.” Trends Biotechnol. 18, 147-151 (2000); Deamer, D. and D. Branton, “Characterization of nucleic acids by nanopore analysis”. Acc. Chem. Res. 35:917-925 (2002); Li, J., M. Gershow, D. Stein, E. Brandin, and J. A. Golovchenko, “DNA molecules and configurations in a solid-state nanopore microscope” Nat. Mater. 2:611-615 (2003), the disclosures of which are incorporated herein by reference in their entireties). In such embodiments, the target nucleic acid passes through a nanopore. The nanopore can be a synthetic pore or biological membrane protein, such as α-hemolysin. As the target nucleic acid passes through the nanopore, each base-pair can be identified by measuring fluctuations in the electrical conductance of the pore. (U.S. Pat. No. 7,001,892; Soni, G. V. & Meller, “A. Progress toward ultrafast DNA sequencing using solid-state nanopores.” Clin. Chem. 53, 1996-2001 (2007); Healy, K. “Nanopore-based single-molecule DNA analysis.” Nanomed. 2, 459-481 (2007); Cockroft, S. L., Chu, J., Amorin, M. & Ghadiri, M. R. “A single-molecule nanopore device detects DNA polymerase activity with single-nucleotide resolution.” J. Am. Chem. Soc. 130, 818-820 (2008), the disclosures of which are incorporated herein by reference in their entireties). Data obtained from nanopore sequencing can be stored, processed and analyzed as set forth herein. In particular, the data can be treated as an image in accordance with the exemplary treatment of optical images and other images that is set forth herein.

Some embodiments can utilize methods involving the real-time monitoring of DNA polymerase activity. Nucleotide incorporations can be detected through fluorescence resonance energy transfer (FRET) interactions between a fluorophore-bearing polymerase and 7-phosphate-labeled nucleotides as described, for example, in U.S. Pat. Nos. 7,329,492 and 7,211,414 (each of which is incorporated herein by reference) or nucleotide incorporations can be detected with zero-mode waveguides as described, for example, in U.S. Pat. No. 7,315,019 (which is incorporated herein by reference) and using fluorescent nucleotide analogs and engineered polymerases as described, for example, in U.S. Pat. No. 7,405,281 and U.S. Patent Application Publication No. 2008/0108082 (each of which is incorporated herein by reference). The illumination can be restricted to a zeptoliter-scale volume around a surface-tethered polymerase such that incorporation of fluorescently labeled nucleotides can be observed with low background (Levene, M. J. et al. “Zero-mode waveguides for single-molecule analysis at high concentrations.” Science 299, 682-686 (2003); Lundquist, P. M. et al. “Parallel confocal detection of single molecules in real time.” Opt. Lett. 33, 1026-1028 (2008); Korlach, J. et al. “Selective aluminum passivation for targeted immobilization of single DNA polymerase molecules in zero-mode waveguide nano structures.” Proc. Natl. Acad. Sci. USA 105, 1176-1181 (2008), the disclosures of which are incorporated herein by reference in their entireties). Images obtained from such methods can be stored, processed and analyzed as set forth herein.

Some SBS embodiments include detection of a proton released upon incorporation of a nucleotide into an extension product. For example, sequencing based on detection of released protons can use an electrical detector and associated techniques that are commercially available from Ion Torrent (Guilford, CT, a Life Technologies subsidiary) or sequencing methods and systems described in US 2009/0026082 A1; US 2009/0127589 A1; US 2010/0137143 A1; or US 2010/0282617 A1, each of which is incorporated herein by reference. Methods set forth herein for amplifying target nucleic acids using kinetic exclusion can be readily applied to substrates used for detecting protons. More specifically, methods set forth herein can be used to produce clonal populations of amplicons that are used to detect protons.

The above SBS methods can be advantageously carried out in multiplex formats such that multiple different target nucleic acids are manipulated simultaneously. In particular embodiments, different target nucleic acids can be treated in a common reaction vessel or on a surface of a particular substrate. This allows convenient delivery of sequencing reagents, removal of unreacted reagents and detection of incorporation events in a multiplex manner. In embodiments using surface-bound target nucleic acids, the target nucleic acids can be in an array format. In an array format, the target nucleic acids can be typically bound to a surface in a spatially distinguishable manner. The target nucleic acids can be bound by direct covalent attachment, attachment to a bead or other particle or binding to a polymerase or other molecule that is attached to the surface. The array can include a single copy of a target nucleic acid at each site (also referred to as a feature) or multiple copies having the same sequence can be present at each site or feature. Multiple copies can be produced by amplification methods such as, bridge amplification or emulsion PCR as described in further detail below.

The methods set forth herein can use arrays having features at any of a variety of densities including, for example, at least about 10 features/cm², 100 features/cm², 500 features/cm², 1,000 features/cm², 5,000 features/cm², 10,000 features/cm², 50,000 features/cm², 100,000 features/cm², 1,000,000 features/cm², 5,000,000 features/cm², or higher.

An advantage of the methods set forth herein is that they provide for rapid and efficient detection of a plurality of target nucleic acid in parallel. Accordingly the present disclosure provides integrated systems capable of preparing and detecting nucleic acids using techniques known in the art such as those exemplified above. Thus, an integrated system of the present disclosure can include fluidic components capable of delivering amplification reagents and/or sequencing reagents to one or more immobilized DNA fragments, the system comprising components such as pumps, valves, reservoirs, fluidic lines and the like. A flow cell can be configured and/or used in an integrated system for detection of target nucleic acids. Exemplary flow cells are described, for example, in US 2010/0111768 A1 and U.S. Ser. No. 13/273,666, each of which is incorporated herein by reference. As exemplified for flow cells, one or more of the fluidic components of an integrated system can be used for an amplification method and for a detection method. Taking a nucleic acid sequencing embodiment as an example, one or more of the fluidic components of an integrated system can be used for an amplification method set forth herein and for the delivery of sequencing reagents in a sequencing method such as those exemplified above. Alternatively, an integrated system can include separate fluidic systems to carry out amplification methods and to carry out detection methods. Examples of integrated sequencing systems that are capable of creating amplified nucleic acids and also determining the sequence of the nucleic acids include, without limitation, the MiSeg™ platform (Illumina, Inc., San Diego, CA) and devices described in U.S. Ser. No. 13/273,666, which is incorporated herein by reference.

The sequencing system described above sequences nucleic-acid polymers present in samples received by a sequencing device. As defined herein, “sample” and its derivatives, is used in its broadest sense and includes any specimen, culture and the like that is suspected of including a target. In some embodiments, the sample comprises DNA, RNA, PNA, LNA, chimeric or hybrid forms of nucleic acids. The sample can include any biological, clinical, surgical, agricultural, atmospheric or aquatic-based specimen containing one or more nucleic acids. The term also includes any isolated nucleic acid sample such a genomic DNA, fresh-frozen or formalin-fixed paraffin-embedded nucleic acid specimen. It is also envisioned that the sample can be from a single individual, a collection of nucleic acid samples from genetically related members, nucleic acid samples from genetically unrelated members, nucleic acid samples (matched) from a single individual such as a tumor sample and normal tissue sample, or sample from a single source that contains two distinct forms of genetic material such as maternal and fetal DNA obtained from a maternal subject, or the presence of contaminating bacterial DNA in a sample that contains plant or animal DNA. In some embodiments, the source of nucleic acid material can include nucleic acids obtained from a newborn, for example as typically used for newborn screening.

The nucleic acid sample can include high molecular weight material such as genomic DNA (gDNA). The sample can include low molecular weight material such as nucleic acid molecules obtained from FFPE or archived DNA samples. In another embodiment, low molecular weight material includes enzymatically or mechanically fragmented DNA. The sample can include cell-free circulating DNA. In some embodiments, the sample can include nucleic acid molecules obtained from biopsies, tumors, scrapings, swabs, blood, mucus, urine, plasma, semen, hair, laser capture micro-dissections, surgical resections, and other clinical or laboratory obtained samples. In some embodiments, the sample can be an epidemiological, agricultural, forensic or pathogenic sample. In some embodiments, the sample can include nucleic acid molecules obtained from an animal such as a human or mammalian source. In another embodiment, the sample can include nucleic acid molecules obtained from a non-mammalian source such as a plant, bacteria, virus or fungus. In some embodiments, the source of the nucleic acid molecules may be an archived or extinct sample or species.

Further, the methods and compositions disclosed herein may be useful to amplify a nucleic acid sample having low-quality nucleic acid molecules, such as degraded and/or fragmented genomic DNA from a forensic sample. In one embodiment, forensic samples can include nucleic acids obtained from a crime scene, nucleic acids obtained from a missing persons DNA database, nucleic acids obtained from a laboratory associated with a forensic investigation or include forensic samples obtained by law enforcement agencies, one or more military services or any such personnel. The nucleic acid sample may be a purified sample or a crude DNA containing lysate, for example derived from a buccal swab, paper, fabric or other substrate that may be impregnated with saliva, blood, or other bodily fluids. As such, in some embodiments, the nucleic acid sample may comprise low amounts of, or fragmented portions of DNA, such as genomic DNA. In some embodiments, target sequences can be present in one or more bodily fluids including but not limited to, blood, sputum, plasma, semen, urine and serum. In some embodiments, target sequences can be obtained from hair, skin, tissue samples, autopsy or remains of a victim. In some embodiments, nucleic acids including one or more target sequences can be obtained from a deceased animal or human. In some embodiments, target sequences can include nucleic acids obtained from non-human DNA such a microbial, plant or entomological DNA. In some embodiments, target sequences or amplified target sequences are directed to purposes of human identification. In some embodiments, the disclosure relates generally to methods for identifying characteristics of a forensic sample. In some embodiments, the disclosure relates generally to human identification methods using one or more target specific primers disclosed herein or one or more target specific primers designed using the primer design criteria outlined herein. In one embodiment, a forensic or human identification sample containing at least one target sequence can be amplified using any one or more of the target-specific primers disclosed herein or using the primer criteria outlined herein.

The components of the customized genotype-imputation system 104 can include software, hardware, or both. For example, the components of the customized genotype-imputation system 104 can include one or more instructions stored on a computer-readable storage medium and executable by processors of one or more computing devices (e.g., the user client device 108, the client device 600). When executed by the one or more processors, the computer-executable instructions of the customized genotype-imputation system 104 can cause the computing devices to perform the bubble detection methods described herein. Alternatively, the components of the customized genotype-imputation system 104 can comprise hardware, such as special purpose processing devices to perform a certain function or group of functions. Additionally, or alternatively, the components of the customized genotype-imputation system 104 can include a combination of computer-executable instructions and hardware.

Furthermore, the components of the customized genotype-imputation system 104 performing the functions described herein with respect to the customized genotype-imputation system 104 may, for example, be implemented as part of a stand-alone application, as a module of an application, as a plug-in for applications, as a library function or functions that may be called by other applications, and/or as a cloud-computing model. Thus, components of the customized genotype-imputation system 104 may be implemented as part of a stand-alone application on a personal computing device or a mobile device. Additionally, or alternatively, the components of the customized genotype-imputation system 104 may be implemented in any application that provides sequencing services including, but not limited to Illumina BaseSpace, Illumina DRAGEN, Illumina TruSight software, ExpansionHunter, or Graph ExpansionHunter. “Illumina,” “BaseSpace,” “DRAGEN,” “TruSight,” “ExpansionHunter,” and “Graph ExpansionHunter” are either registered trademarks or trademarks of Illumina, Inc. in the United States and/or other countries.

Embodiments of the present disclosure may comprise or utilize a special purpose or general-purpose computer including computer hardware, such as, for example, one or more processors and system memory, as discussed in greater detail below. Embodiments within the scope of the present disclosure also include physical and other computer-readable media for carrying or storing computer-executable instructions and/or data structures. In particular, one or more of the processes described herein may be implemented at least in part as instructions embodied in a non-transitory computer-readable medium and executable by one or more computing devices (e.g., any of the media content access devices described herein). In general, a processor (e.g., a microprocessor) receives instructions, from a non-transitory computer-readable medium, (e.g., a memory, etc.), and executes those instructions, thereby performing one or more processes, including one or more of the processes described herein.

Computer-readable media can be any available media that can be accessed by a general purpose or special purpose computer system. Computer-readable media that store computer-executable instructions are non-transitory computer-readable storage media (devices). Computer-readable media that carry computer-executable instructions are transmission media. Thus, by way of example, and not limitation, embodiments of the disclosure can comprise at least two distinctly different kinds of computer-readable media: non-transitory computer-readable storage media (devices) and transmission media.

Non-transitory computer-readable storage media (devices) includes RAM, ROM, EEPROM, CD-ROM, solid state drives (SSDs) (e.g., based on RAM), Flash memory, phase-change memory (PCM), other types of memory, other optical disk storage, magnetic disk storage or other magnetic storage devices, or any other medium which can be used to store desired program code means in the form of computer-executable instructions or data structures and which can be accessed by a general purpose or special purpose computer.

A “network” is defined as one or more data links that enable the transport of electronic data between computer systems and/or modules and/or other electronic devices. When information is transferred or provided over a network or another communications connection (either hardwired, wireless, or a combination of hardwired or wireless) to a computer, the computer properly views the connection as a transmission medium. Transmissions media can include a network and/or data links which can be used to carry desired program code means in the form of computer-executable instructions or data structures and which can be accessed by a general purpose or special purpose computer. Combinations of the above should also be included within the scope of computer-readable media.

Further, upon reaching various computer system components, program code means in the form of computer-executable instructions or data structures can be transferred automatically from transmission media to non-transitory computer-readable storage media (devices) (or vice versa). For example, computer-executable instructions or data structures received over a network or data link can be buffered in RAM within a network interface module (e.g., a NIC), and then eventually transferred to computer system RAM and/or to less volatile computer storage media (devices) at a computer system. Thus, it should be understood that non-transitory computer-readable storage media (devices) can be included in computer system components that also (or even primarily) utilize transmission media.

Computer-executable instructions comprise, for example, instructions and data which, when executed at a processor, cause a general purpose computer, special purpose computer, or special purpose processing device to perform a certain function or group of functions. In some embodiments, computer-executable instructions are executed on a general-purpose computer to turn the general-purpose computer into a special purpose computer implementing elements of the disclosure. The computer executable instructions may be, for example, binaries, intermediate format instructions such as assembly language, or even source code. Although the subject matter has been described in language specific to structural features and/or methodological acts, it is to be understood that the subject matter defined in the appended claims is not necessarily limited to the described features or acts described above. Rather, the described features and acts are disclosed as example forms of implementing the claims.

Those skilled in the art will appreciate that the disclosure may be practiced in network computing environments with many types of computer system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, tablets, pagers, routers, switches, and the like. The disclosure may also be practiced in distributed system environments where local and remote computer systems, which are linked (either by hardwired data links, wireless data links, or by a combination of hardwired and wireless data links) through a network, both perform tasks. In a distributed system environment, program modules may be located in both local and remote memory storage devices.

Embodiments of the present disclosure can also be implemented in cloud computing environments. In this description, “cloud computing” is defined as a model for enabling on-demand network access to a shared pool of configurable computing resources. For example, cloud computing can be employed in the marketplace to offer ubiquitous and convenient on-demand access to the shared pool of configurable computing resources. The shared pool of configurable computing resources can be rapidly provisioned via virtualization and released with low management effort or service provider interaction, and then scaled accordingly.

A cloud-computing model can be composed of various characteristics such as, for example, on-demand self-service, broad network access, resource pooling, rapid elasticity, measured service, and so forth. A cloud-computing model can also expose various service models, such as, for example, Software as a Service (SaaS), Platform as a Service (PaaS), and Infrastructure as a Service (IaaS). A cloud-computing model can also be deployed using different deployment models such as private cloud, community cloud, public cloud, hybrid cloud, and so forth. In this description and in the claims, a “cloud-computing environment” is an environment in which cloud computing is employed.

FIG. 12 illustrates a block diagram of a computing device 1200 that may be configured to perform one or more of the processes described above. One will appreciate that one or more computing devices such as the computing device 1200 may implement the customized genotype-imputation system 104 and the sequencing system 106. As shown by FIG. 12, the computing device 1200 can comprise a processor 1202, a memory 1204, a storage device 1206, an I/O interface 1208, and a communication interface 1210, which may be communicatively coupled by way of a communication infrastructure 1212. In certain embodiments, the computing device 1200 can include fewer or more components than those shown in FIG. 12. The following paragraphs describe components of the computing device 1200 shown in FIG. 12 in additional detail.

In one or more embodiments, the processor 1202 includes hardware for executing instructions, such as those making up a computer program. As an example, and not by way of limitation, to execute instructions for dynamically modifying workflows, the processor 1202 may retrieve (or fetch) the instructions from an internal register, an internal cache, the memory 1204, or the storage device 1206 and decode and execute them. The memory 1204 may be a volatile or non-volatile memory used for storing data, metadata, and programs for execution by the processor(s). The storage device 1206 includes storage, such as a hard disk, flash disk drive, or other digital storage device, for storing data or instructions for performing the methods described herein.

The I/O interface 1208 allows a user to provide input to, receive output from, and otherwise transfer data to and receive data from computing device 1200. The I/O interface 1208 may include a mouse, a keypad or a keyboard, a touch screen, a camera, an optical scanner, network interface, modem, other known I/O devices or a combination of such I/O interfaces. The I/O interface 1208 may include one or more devices for presenting output to a user, including, but not limited to, a graphics engine, a display (e.g., a display screen), one or more output drivers (e.g., display drivers), one or more audio speakers, and one or more audio drivers. In certain embodiments, the I/O interface 1208 is configured to provide graphical data to a display for presentation to a user. The graphical data may be representative of one or more graphical user interfaces and/or any other graphical content as may serve a particular implementation.

The communication interface 1210 can include hardware, software, or both. In any event, the communication interface 1210 can provide one or more interfaces for communication (such as, for example, packet-based communication) between the computing device 1200 and one or more other computing devices or networks. As an example, and not by way of limitation, the communication interface 1210 may include a network interface controller (NIC) or network adapter for communicating with an Ethernet or other wire-based network or a wireless NIC (WNIC) or wireless adapter for communicating with a wireless network, such as a WI-FI.

Additionally, the communication interface 1210 may facilitate communications with various types of wired or wireless networks. The communication interface 1210 may also facilitate communications using various communication protocols. The communication infrastructure 1212 may also include hardware, software, or both that couples components of the computing device 1200 to each other. For example, the communication interface 1210 may use one or more networks and/or protocols to enable a plurality of computing devices connected by a particular infrastructure to communicate with each other to perform one or more aspects of the processes described herein. To illustrate, the sequencing process can allow a plurality of devices (e.g., a client device, sequencing device, and server device(s)) to exchange information such as sequencing data and error notifications.

In the foregoing specification, the present disclosure has been described with reference to specific exemplary embodiments thereof. Various embodiments and aspects of the present disclosure(s) are described with reference to details discussed herein, and the accompanying drawings illustrate the various embodiments. The description above and drawings are illustrative of the disclosure and are not to be construed as limiting the disclosure. Numerous specific details are described to provide a thorough understanding of various embodiments of the present disclosure.

The present disclosure may be embodied in other specific forms without departing from its spirit or essential characteristics. The described embodiments are to be considered in all respects only as illustrative and not restrictive. For example, the methods described herein may be performed with less or more steps/acts or the steps/acts may be performed in differing orders. Additionally, the steps/acts described herein may be repeated or performed in parallel with one another or in parallel with different instances of the same or similar steps/acts. The scope of the present application is, therefore, indicated by the appended claims rather than by the foregoing description. All changes that come within the meaning and range of equivalency of the claims are to be embraced within their scope.

What is claimed is:

1. A computer-implemented method comprising:
generating a reference panel comprising marker-variant indicators for marker variants at genomic coordinates corresponding to genomic samples of different haplotypes;
adding at least one target-variant position to the reference panel indicating a presence or absence of a target variant within the genomic samples;
phasing, based on the marker variants, alleles of the genomic samples to determine a presence or absence of the target variant in corresponding alleles present on maternal haplotypes and paternal haplotypes; and
generating a target-variant-reference panel comprising target-variant indicators within the at least one target-variant position for the phased alleles of the genomic samples.

2. The computer-implemented method of claim 1, wherein the at least one target-variant position comprises a target-variant position for target-variant indicators of a biallelic target variant or a multi-allelic target variant.

3. The computer-implemented method of claim 1, wherein phasing the alleles of the genomic samples comprises phasing heterozygous alleles of a subset of the genomic samples.

4. The computer-implemented method of claim 1, wherein the marker variants comprise single-nucleotide polymorphisms (SNPs).

5. The computer-implemented method of claim 1, wherein generating the reference panel comprises generating a phased reference panel comprising the marker-variant indicators for marker variants phased according to the maternal haplotypes and the paternal haplotypes of the genomic samples.

6. The computer-implemented method of claim 1, wherein the target variant comprises a repeat expansion.

7. The computer-implemented method of claim 1, wherein the target variant comprises a deletion, an insertion, a duplication, an inversion, a translocation, or a copy number variation (CNV) transmitted within a population.

8. The computer-implemented method of claim 1, wherein the target variant satisfies one or more of a threshold carrier frequency, a threshold linkage disequilibrium (LD) with respect to particular marker variants, or a threshold mutation rate.

9. A system comprising:
at least one processor; and
a non-transitory computer readable medium comprising instructions that, when executed by the at least one processor, cause the system to:
identify nucleotide reads corresponding to a target genomic sample;
access a target-variant-reference panel comprising target-variant indicators within at least one target-variant position for phased alleles of genomic samples of different haplotypes; and
impute a genotype call for a target variant within the target genomic sample based on a comparison of the target-variant-reference panel and the nucleotide reads corresponding to the target genomic sample.

10. The system of claim 9, wherein the target-variant indicators indicate a presence or absence of the target variant in the at least one target-variant position for the phased alleles of the genomic samples.

11. The system of claim 9, further comprising instructions that, when executed by the at least one processor, cause the system to:
determine phased alleles of the target genomic sample based on the comparison of the target-variant-reference panel and the nucleotide reads corresponding to the target genomic sample; and
impute the genotype call by imputing a phased genotype call for the target variant within the target genomic sample based on the phased alleles of the target genomic sample.

12. The system of claim 9, further comprising instructions that, when executed by the at least one processor, cause the system to impute the genotype call for the target variant by generating a prediction of whether the target genomic sample comprises the target variant.

13. The system of claim 12, further comprising instructions that, when executed by the at least one processor, cause the system to generate the prediction by predicting whether the target genomic sample comprises a pathogenic variant at an allele present on a maternal haplotype or a paternal haplotype.

14. The system of claim 9, further comprising instructions that, when executed by the at least one processor, cause the system to impute the genotype call by:
identifying, within the nucleotide reads corresponding to the target genomic sample, one or more single-nucleotide polymorphisms (SNPs) as one or more marker variants within the target-variant-reference panel for the target variant; and
determining the genotype call further based on the one or more SNPs within the nucleotide reads.

15. The system of claim 9, further comprising instructions that, when executed by the at least one processor, cause the system to impute the genotype call for the target variant by imputing the genotype call for a repeat expansion.

16. A non-transitory computer-readable medium storing instructions that, when executed by at least one processor, cause a computing device to:
identify nucleotide reads corresponding to a target genomic sample;
access a target-variant-reference panel comprising target-variant indicators within at least one target-variant position for phased alleles of genomic samples; and
impute a genotype call for a target variant within the target genomic sample based on a comparison of the target-variant-reference panel and the nucleotide reads corresponding to the target genomic sample.

17. The non-transitory computer-readable medium of claim 16, wherein the target-variant indicators indicate a presence or absence of the target variant in the at least one target-variant position for the phased alleles of genomic samples.

18. The non-transitory computer-readable medium of claim 16, further comprising instructions that, when executed by the at least one processor, cause the computing device to:
determine phased alleles for the target genomic sample based on the comparison of the target-variant-reference panel and the nucleotide reads corresponding to the target genomic sample; and
impute the genotype call by imputing a phased genotype call for the target variant within the target genomic sample based on the phased alleles of the target genomic sample.

19. The non-transitory computer-readable medium of claim 16, further comprising instructions that, when executed by the at least one processor, causes the computing device to impute the genotype call for the target variant by imputing the genotype call for a Replication Factor C Subunit 1 (RFC1) gene, a Cytochrome P450 Family 2 Subfamily D Member 6 (CYP2D6) gene, Cytochrome P450 Family 2 Subfamily B Member 6 (CYP2B6) gene, Cytochrome P450 Family 21 Subfamily A Member 2 (CYP21A2) gene, Survival Motor Neuron 1 (SMN1) gene, Survival Motor Neuron 2 (SMN2) gene, Glucosylceramides Beta (GBA) gene, Blood Group Rh(CE) (RHCE) gene, Lipoprotein(A) (LPA) gene, a Fragile X Mental Retardation 1 (FMR1) gene, a Hexosaminidase Subunit Alpha (HEXA) gene, Hemoglobin Subunit Alpha 1 (HBA1) gene, Hemoglobin Subunit Alpha 2 (HBA2) gene, or a Hemoglobin Subunit Beta (HBB) gene.

20. The non-transitory computer-readable medium of claim 16, wherein the target-variant-reference panel comprises marker-variant indicators for marker variants within a target genomic region for the target variant and does not comprise additional marker-variant indicators for additional marker variants outside of the target genomic region.
